(19) United States
(12) Patent Application Publication
(10) Pub. No.: US 2018/0135074 A1
(43) Pub. Date: May 17, 2018

(54) RECOMBINANT AAV VECTORS FOR GENE THERAPY OF HUMAN HEMATOPOIETIC DISORDERS

(71) Applicant: University of Florida Research Foundation, Incorporated, Gainesville, FL (US)

(72) Inventors: Arun Srivastava, Gainesville, FL (US); Chen Ling, Gainesville, FL (US); Mavis Agbandje-McKenna, Gainesville, FL (US)

(73) Assignee: University of Florida Research Foundation, Incorporated, Gainesville, FL (US)

(21) Appl. No.: 15/552,127

(22) PCT Filed: Feb. 19, 2016
(86) PCT No.: PCT/US16/18815

§ 371 Date: Aug. 18, 2017

Related U.S. Application Data
(60) Provisional application No. 62/118,095, filed on Feb. 19, 2015.
(60) Provisional application No. 62/118,139, filed on Feb. 19, 2015.
(60) Provisional application No. 62/118,114, filed on Feb. 19, 2015.

(51) Int. Cl. C12N 15/85; C07K 14/805; C12N 15/864; A61K 35/76; C07K 14/47; A61K 48/00; A61P 7/00

(52) U.S. Cl. CPC C12N 15/8509; C07K 14/805; C12N 15/864; A61K 35/76; C07K 14/4717; C12N 2830/008; A61P 7/00; C12N 2750/14122; C12N 2750/14243; C12N 2750/14143; A61K 48/00

(57) ABSTRACT

Provided herein are recombinant AAV (rAAV) particles comprising a nucleic acid vector comprising a parvovirus B 19p6 promoter operatively linked to a heterologous gene, such as a human globin gene, and rAAV capsid proteins comprising one or more amino acid substitutions in a surface exposed loop of the capsid protein that result, e.g., in increased P antigen binding compared to a corresponding un-mutated AAV capsid protein. Also provided are methods and compositions related to such capsid proteins, methods of targeting gene expression to a cell of erythroid lineage, methods of treating a hemoglobinopathy using such rAAV particles, and methods for efficient transduction of a host cell suspension with a rAAV.

### RELATED APPLICATIONS

This application claims the benefit under 35 U.S.C. § 119(e) of U.S. provisional application No. 62/118,095, filed Feb. 19, 2015, U.S. provisional application No. 62/118,139, filed Feb. 19, 2015, and U.S. provisional application No. 62/118,114, filed Feb. 19, 2015, the contents of each of which are incorporated herein by reference in their entirety.

### GOVERNMENT SUPPORT

This invention was made with government support under HL-097088 and EB-015684 awarded by the National Institutes of Health. The government has certain rights in the invention.

### BACKGROUND

Different recombinant adeno-associated virus (rAAV) serotypes have tropisms for different tissues and cell types. There remains a need to develop serotypes that can selectively target tissues and cell types of interest. Additionally, methods for infecting cells with recombinant adeno-associated virus (rAAV) remain limited in their ability to be efficient in certain cell types. Accordingly, methods are needed to address the limited infection rates in those cells.

### SUMMARY

Aspects of the application relate to compositions and methods for treating disorders relating to the hematopoietic system using recombinant AAV (rAAV). Aspects of the application include cell-specific expression, cell-specific targeting, efficient rAAV transduction, and combinations thereof.

In some aspects, the application provides rAAV particles and nucleic acid vectors that comprise a parvovirus B19p6 promoter operatively linked to a heterologous gene, such as a human globin gene. Also provided are various methods that utilize such particles and nucleic acid vectors, such as methods of treating hemoglobinopathies.

In some aspects, the disclosure provides an rAAV particle comprising a nucleic acid vector comprising a parvovirus B19p6 promoter operatively linked to a heterologous gene. In some embodiments, the rAAV particle is not AAV2. In some embodiments, the rAAV particle is AAV2. In some embodiments, the rAAV particle is AAV6.

In some embodiments, the heterologous gene is a globin gene. In some embodiments, the globin gene is selected from the group consisting of a β-globin gene, an anti-sickling β-globin gene, and a γ-globin gene. In some embodiments, the globin gene is a human globin gene. In some embodiments, the globin gene is a human β-globin gene or human anti-sickling β-globin gene.

In some embodiments, the rAAV particle is a AAV6 particle. In some embodiments, the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination of two or more thereof.

In some embodiments, the modified capsid protein comprises a non-tyrosine residue and/or a non-threonine residue at one or more of or each of Y705, Y731, and T492 of a wild-type AAV6 capsid protein. In some embodiments, the non-tyrosine residue is phenylalanine and the non-threonine residue is valine.

In some embodiments, the nucleic acid vector further comprises AAV2 or AAV6 inverted terminal repeat sequences (ITRs) flanking the parvovirus B19p6 promoter operatively linked to the heterologous gene.

Other aspects of the disclosure relate to a nucleic acid vector comprising a parvovirus B19p6 promoter operatively linked to a globin gene. In some embodiments, the globin gene is selected from the group consisting of a β-globin gene, an anti-sickling β-globin gene, and a γ-globin gene. In some embodiments, the globin gene is a human globin gene. In some embodiments, the globin gene is a human β-globin gene or human anti-sickling β-globin gene.

Further aspects of the disclosure relate to rAAV capsid proteins comprising one or more amino acid substitutions in a surface-exposed loop region. In some embodiments, the substitutions result in improved targeting of a tissue or cell of interest, e.g., a cell expressing P antigen.

Aspects of the disclosure relate to an rAAV capsid protein comprising one or more amino acid substitutions that result in increased P antigen binding compared to a corresponding un-mutated AAV capsid protein, wherein the one or more amino acid substitutions are in a surface exposed loop of the capsid protein. Other aspects of the disclosure relate to an rAAV capsid protein comprising one or more amino acid substitutions that introduce a P antigen binding site into a surface exposed loop of the capsid protein. In some embodiments, the surface exposed loop is loop VIII. In some embodiments, a surface exposed loop is replaced by a B19 P antigen binding site. In some embodiments, the B19 P antigen binding site comprises the amino acid sequence QQYTDQIE (SEQ ID NO: 1). In some embodiments, the rAAV capsid protein is a variant of an AAV6 capsid protein.

Other aspects of the disclosure provide a method of increasing rAAV tropism for hematopoietic stem cells, the method comprising altering a surface exposed loop of an AAV capsid protein to introduce one or more amino acid substitutions that result in increased P antigen binding compared to a corresponding un-mutated AAV capsid protein. Yet other aspects of the disclosure provide a method of increasing rAAV tropism for hematopoietic stem cells, the method comprising altering a surface exposed loop of an AAV capsid protein to introduce one or more amino acid substitutions that introduce a P antigen binding site into a surface exposed loop of the capsid protein. In some embodiments, the surface exposed loop is loop VIII. In some embodiments, a surface exposed loop is replaced by a B19 P antigen binding site. In some embodiments, the B19 P antigen binding site comprises the amino acid sequence QQYTDQIE (SEQ ID NO: 1). In some embodiments, the rAAV capsid protein is a variant of an AAV6 capsid protein.

Other aspects of the disclosure relate to a method of delivering an rAAV to a cell, the method comprising administering an rAAV particle comprising an rAAV capsid protein of any one of the embodiments above or described herein. In some embodiments, the cell is a hematopoietic stem cell, a megakaryocyte, an endothelial cell, a cardiomyocyte, a hepatocyte, or a trophoblast. In some embodiments, the cell is a hematopoietic stem cell. In some embodiments, the subject is a human subject.

Other aspects of the disclosure relate to an rAAV particle comprising an rAAV capsid protein of any one of the embodiments above or described herein.

Yet further aspects of the disclosure relate to a nucleic acid encoding an rAAV capsid protein of any one of the embodiments above or described herein. In some embodiments, the nucleic acid is a plasmid.

In some aspects, the disclosure provides an rAAV particle comprising a nucleic acid vector comprising a parvovirus B19p6 promoter operatively linked to a heterologous gene, wherein the rAAV particle capsid protein comprises one or more amino acid substitutions that result in increased P antigen binding compared to a corresponding un-mutated AAV capsid protein.

Other aspects of the disclosure relate to a method of targeting gene expression to a cell of erythroid lineage in a subject, the method comprising administering the rAAV particle of any one of the embodiments above or described herein or the nucleic acid vector of any one of the embodiments above or described herein to a subject. In some embodiments, the subject is a human subject. In some embodiments, the cell of erythroid lineage is a hematopoietic stem cell. In some embodiments, the cell of erythroid lineage is a CD36+ burst-forming units-erythroid (BFU-E) cell or a colony-forming unit-erythroid (CFUE-E) progenitor cell.

Yet other aspects of the disclosure relate to a method of treating a hemoglobinopathy, the method comprising administering the rAAV particle of any one of the embodiments above or described herein or the nucleic acid vector of any one of the embodiments above or described herein to a subject having a hemoglobinopathy. In some embodiments, the subject is a human subject. In some embodiments, the hemoglobinopathy is β-thalassemia or sickle cell disease.

Also provided herein are methods of achieving efficient rAAV transduction of host cells that are grown suspension, such as hematopoietic stem and progenitor cells (e.g., bone marrow-derived cells, cord blood-derived cells, CD34+ cells, and CD36+ cells) and other cell types that are grown under non-adherent conditions. As described herein, it has been shown that cell suspensions grown at high density (e.g., at 200,000 cell per 50 microliters or greater) showed improved transduction efficiency of rAAV particles compared to cell suspensions grown at low densities.

In some aspects, the disclosure provides a method for efficient AAV transduction of a host cell suspension, the method comprising contacting a host cell suspension with a recombinant AAV (rAAV) particle composition, wherein the host cell suspension has a density of greater than 4,000 cells per microliter.

In some embodiments, the rAAV particle composition is a AAV2 or AAV6 particle composition. In some embodiments, the recombinant AAV (rAAV) particle within the composition comprises a modified capsid protein comprising a non-tyrosine residue at a position that corresponds to a surface-exposed tyrosine residue in a wild-type AAV2 or AAV6 capsid protein, a non-threonine residue at a position that corresponds to a surface-exposed threonine residue in the wild-type AAV2 or AAV6 capsid protein, a non-lysine residue at a position that corresponds to a surface-exposed lysine residue in the wild-type AAV2 or AAV6 capsid protein, a non-serine residue at a position that corresponds to a surface-exposed serine residue in the wild-type AAV2 or AAV6 capsid protein, or a combination thereof. In some embodiments, the modified capsid protein comprises a non-tyrosine residue and/or a non-threonine residue at one or more of or each of Y705, Y731, and T492 of a wild-type AAV6 capsid protein. In some embodiments, the modified capsid protein comprises a non-tyrosine residue and/or a non-threonine residue at one or more of or each of Y444, Y500, Y731, and T491 of a wild-type AAV2 capsid protein. In some embodiments, the non-tyrosine residue is phenylalanine and the non-threonine residue is valine.

In some embodiments, the rAAV particle composition contains 3×10³-1×10⁴ vector genomes (vg)/mL of rAAV particles. In some embodiments, the rAAV particle composition contains 1×10²-1×10⁶, 1×10³-1×10⁶, 1×10³-1×10⁵, or 1×10³-1×10⁴ vg/mL of rAAV particles.

In some embodiments, the recombinant AAV (rAAV) particle within the composition comprises a nucleic acid vector that encodes a therapeutic protein.

In some embodiments, the rAAV particle within the composition comprises a nucleic acid vector comprising a parvovirus B19p6 promoter operatively linked to a heterologous gene. In some embodiments, the rAAV particle within the composition comprises an rAAV capsid protein comprising one or more amino acid substitutions that result in increased P antigen binding compared to a corresponding un-mutated AAV capsid protein.

In some embodiments, the host cell suspension comprises stem cells. In some embodiments, the host cell suspension comprises human cells. In some embodiments, the host cell suspension comprises hematopoietic stem cells. In some embodiments, the host cell suspension is a non-adherent host cell suspension. In some embodiments, the method further comprises administering host cells of the host cell suspension to a subject. In some embodiments, host cells of the host cell suspension are obtained from a subject.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a non-limiting illustration of recombinant AAV nucleic acid vectors.

FIG. 2 shows a structural alignment of loop VIII of an AAV6 capsid protein with the P antigen binding site. The sequences from top to bottom correspond to SEQ ID NOs.: 1 and 28, respectively.

FIG. 3 is a non-limiting schematic representation of AAV vector-mediated transduction of HEK293 (3A, 3C), K562 (3B, 3D), M07e (3E, 3F), and Raji (3G, 3H) cells at low and high cell densities, respectively.

FIG. 4 shows non-limiting results of transductions efficiencies of rAAV2 and rAAV6 particles at 3×10³-1×10⁴ vector genomes(vgs)/cell at low (20,000 or 60, 000 cells in 50 microliters) or high (200,000 or 580,000 cells in 50 microliters) cell densities. The particles tested contained wild-type (WT) or mutated capsid proteins (for AAV2: Capsid-modified quadruple-mutant (4444F+Y500F+Y731F+T491V) and for AAV6: Capsid-modified triple-mutant (Y705F+Y731F+T492V)).

FIG. 5 shows non-limiting results of transduction efficiencies of AAV in human hematopoietic cells at various cell densities. K562 cells were transduced at various indicated cell densities at MOIs of 3,000 or 30,000 vgs/mL with WT scAAV6-CBAp-EGFP (5A). K562 cells were also transduced at low or high cell densities with TM scAAV6-CBAp-EGFP (5B). The vector genome copy numbers/cell were determined 2 hours post-transduction by qPCR and data were normalized to β-actin DNA copy number (5C). K562 cells were transduced at low or high cell densities with TM scAAV6-CBAp-Gluc, and transgene expression and mean fluorescence intensity were determined in the culture supernatants (5D). K562 cells were transduced at low or high cell densities with QM scAAV2-CBAp-EGFP (5E). Primary human bone marrow-derived CD34+ cells were transduced at low or high densities with indicated AA6 or AAV2, EGFP-expressing cells were visualized under a fluorescence microscope 48 hours post-transduction (5F). The vector genome copy numbers/cell (5G).

FIG. 6 shows non-limiting data depicting the effect of initial cell-cell contact. Schematics of the experiment design (6A). K562 cells and 8 replicate cultures were transduced at low-density (L) with scAAV6-TM-CBAp-EGFP and subsequently pooled together to reach high-density (L→H), and conversely, cells were transduced at high-density (H), and were subsequently diluted to low-density (H→L). Transgene expression in each cell population was analyzed by fluorescence microscopy 48 hours post-transduction (6B). Mean fluorescence intensity of transgene expression is additionally depicted (6C).

FIG. 7 shows non-limiting data depicting the effect of culture volume. Schematics of the experiment design (7A). A fixed number of K562 cells were transduced with viruses in various indicated volumes and subsequently diluted to 2.0 mL. Transgene expression in each cell population was analyzed by fluorescence microscopy (7B). Mean fluorescence intensity of transgene expression is additionally depicted (7C).

FIG. 8 shows non-limiting data depicting the transduction efficiency of AAV in various human hematopoietic cell lines at low and high cell densities. (A) Human K562, M07e, and Raji cells were transduced with scAAV2-CBAp-EGFP at either low or high cell density (8A). Mean fluorescence intensity of transgene expression in each cell type is depicted (8B). FACS analyses of the level of expression of membrane heparin sulfate proteoglycan in various human cell types (8C). Each cell type was transduced with scAAV6-TM-CBAp-EGFP at high cell density, and transgene expression was analyzed 48 hours post-transduction (8D). Mean fluorescence intensity of transgene expression in each cell type is additionally depicted (8E).

### DETAILED DESCRIPTION

In various aspects, the application provides compositions and methods for treating disorders relating to the hematopoietic system with recombinant AAV (rAAV). Aspects of the application include cell-specific expression, cell-specific targeting, efficient rAAV transduction, and combinations thereof. In some aspects, the disclosure provides methods that are useful in the preparation of therapeutic compositions. In some aspects, the disclosure provides compositions that are useful in therapeutic applications. As described herein, such methods and compositions are useful in treating hematopoietic diseases and disorders (e.g., hemoglobinopathies).

In some aspects, the disclosure relates to recombinant AAV (rAAV) particles and nucleic acid vectors that comprise a parvovirus B19p6 promoter operatively linked to a heterologous gene, such as a human globin gene. As described herein, such particles and vectors are useful for targeting cells, such as cells of the erythroid lineage. As used herein, the term “vector” can refer to a nucleic acid vector (e.g., a plasmid or recombinant viral genome) or a viral vector (e.g., an rAAV particle comprising a recombinant genome).

Other aspects of the disclosure relate to targeting gene expression to a cell, such as a cell of erythroid lineage. In some embodiments, the method comprises administering a rAAV particle described herein or a nucleic acid vector described herein to a cell. The administration may be ex vivo (e.g., to a cell in a culture) or in vivo (e.g., in a subject).

In some embodiments, the cell of erythroid lineage is a hematopoietic stem cell. In some embodiments, the hematopoietic stem cell is a CD34⁺, lin⁻ HSC. In some embodiments, the cell of erythroid lineage is a CD36⁺ burst-forming units-erythroid (BFU-E) cell or a colony-forming unit-erythroid (CFUE-E) progenitor cell. In some embodiments, the cells are identified as being CD36⁺ and/or glycophorin A⁺. HSCs and other cell types expressing particular markers, such CD34, lin, CD36, or glycophorin A, can be detected, sorted, and collected using any method known in the art, e.g., by single-cell sorting methods such as fluorescence-activated cell sorting.

Other aspects of the disclosure relate to a method of treating a hemoglobinopathy. In some embodiments, the method comprises administering a rAAV particle described herein or a nucleic acid vector described herein to a subject (e.g., a human subject) having a hemoglobinopathy (e.g., a is β-thalassemia or sickle cell disease).

Other aspects of the disclosure relate to a method of increasing rAAV tropism for a tissue or cell of interest. In some embodiments, the method comprises altering a surface exposed loop of an AAV capsid protein to introduce one or more amino acid substitutions that result in increased binding to a cell or tissue of interest compared to a corresponding un-mutated AAV capsid protein.

In some aspects, the disclosure relates to methods of targeting rAAV particles by modifying one or more surface exposed loops, e.g., by replacing all or part of the loop with a sequence that enhances binding to a cell or tissue of interest. Related compositions, host cells, nucleic acids, and rAAV particles are also provided.

In some aspects, the disclosure relates to an rAAV capsid protein comprising one or more amino acid substitutions are in a surface exposed loop of the capsid protein. In some embodiments, the one or more amino acid substitutions result in increased P antigen binding compared to a corresponding unmutated AAV capsid protein. In some embodiment, a rAAV capsid protein is provided comprising one or more amino acid substitutions that introduce a P antigen binding site into a surface exposed loop of the capsid protein. In some embodiments, the surface exposed loop is any one of loops I to IX. In some embodiments, the surface exposed loop is loop VIII.

In some embodiments, a surface exposed loop is replaced by a B19 P antigen binding site. In some embodiments, the B19 P antigen binding site comprises the amino acid sequence QQYTDQIE, or a fragment or variant thereof that is capable of binding to P antigen. A P antigen binding site can be identified, e.g., by mutagenesis of known P antigen binding sites, e.g., using phage display or site-directed mutagenesis in combination with a binding assay such as a surface plasmon resonance, ELISA, or co-immunoprecipitation assay. In some embodiments, the rAAV capsid protein is an AAV6 capsid protein comprising the one or more amino acid substitutions in a surface exposed loop. In some embodiments, AAV6 loop VIII (residues 592 to 598) are substituted with a P antigen-binding site (e.g., residues 399 to 406, QQYTDQIE, of human parvovirus B19). An exemplary wild-type AAV6 capsid protein is provided below. Loops I-IX are underlined and bolded. Loop VIII is underlined, bolded and italicized.

(SEQ ID NO: 21)

| | |
|---|---|
| 1 | MAADGYLPDW LEDNLSEGIR EWWDLKPGAP KPKANQQKQD |
| | DGRGLVLPGY |
| 51 | KYLGPFNGLD KGEPVNAADA AALEHDKAYD QQLKAGDNPY |
| | LRYNHADAEF |
| 101 | QERLQEDTSF GGNLGRAVFQ AKKRVLEPFG LVEEGAKTAP |
| | GKKRPVEQSP |
| 151 | QEPDSSSGIG KTGQQPAKKR LNFGQTGDSE SVPDPQPLGE |
| | PPATPAAVGP |
| 201 | TTMASGGGAP MADNNEGADG VGNASGNWHC DSTWLGDRVI |
| | TTSTRTWALP |
| 251 | TYNNHLYKQI SSASTGASND NHYFGYSTPW GYFDFNRFHC |
| | HFSPRDWQRL |
| 301 | INNNWGFRPK RLNFKLFNIQ VKEVTTNDGV TTIANNLTST |
| | VQVFSDSEYQ |
| 351 | LPYVLGSAHQ GCLPPFPADV FMIPQYGYLT LNNGSQAVGR |
| | SSFYCLEYFP |
| 401 | SQMLRTGNNF TFSYTFEDVP FHSSYAHSQS LDRLMNPLID |
| | QYLYYLNRTQ |
| 451 | NQSGSAQNKD LLFSRGSPAG MSVQPKNWLP GPCYRQQRVS |
| | KTKTDNNNSN |
| 501 | FTWTGASKYN LNGRESIINP GTAMASHKDD KDKFFPMSGV |
| | MIFGKESAGA |
| 551 | SNTALDNVMI TDEEEIKATN PVATERFGTV AVNLQSSSTD |
| | P [symbol] MG |
| 601 | ALPGMVWQDR DVYLQGPIWA KIPHTDGHFH PSPLMGGFGL |
| | KHPPPQILIK |
| 651 | NTPVPANPPA EFSATKFASF ITQYSTGQVS VEIEWELQKE |
| | NSKRWNPEVQ |
| 701 | YTSNYAKSAN VDFTVDNNGL YTEPRPIGTR YLTRPL |

In some embodiments, the cell of interest is a cell expressing P antigen (e.g., a hematopoietic stem cell), and the method comprises altering a surface exposed loop of an AAV capsid protein to introduce one or more amino acid substitutions that result in increased P antigen binding compared to a corresponding un-mutated AAV capsid protein. In some embodiments, the method comprises altering a surface exposed loop of an AAV capsid protein to introduce one or more amino acid substitutions that introduce a P antigen binding site into a surface exposed loop of the capsid protein. In some embodiments, the surface exposed loop is loop VIII. In some embodiments, the surface exposed loop is replaced by a B19 P antigen binding site. In some embodiments, the B19 P antigen binding site comprises the amino acid sequence QQYTDQIE (SEQ ID NO: 1), or a fragment or variant thereof that is capable of binding to P antigen.

In some embodiments, the rAAV capsid protein is an AAV6 capsid protein comprising the one or more amino acid substitutions in a surface exposed loop. AAV6 capsid proteins are further described herein. In some embodiments, AAV6 loop VIII (residues 592 to 598) is substituted with a P antigen-binding site (e.g., residues 399 to 406, QQYTDQIE (SEQ ID NO: 1), of human parvovirus B19).

Other aspects of the disclosure relate to a method of delivering an rAAV to a cell, the method comprising administering an rAAV particle comprising an rAAV capsid protein as described herein. In some embodiments, the cell is ex vivo. In some embodiments, the cell is in vivo, e.g., in a subject as described herein, such as a human subject.

In some embodiments, the cell is a cell expressing P antigen. A cell expressing P antigen can be identified, e.g., by Western blot, ELISA, or another immunoassay known in the art utilizing a P antigen antibody or antigen-binding fragment thereof. An exemplary human P antigen amino acid sequence is provided below:

(SEQ ID NO: 2)
NP_001033717.1|UDP-GalNAc: beta-1,3-N-acetyl-
galactosaminyltransferase 1 [Homo sapiens]
MASALWTVLPSRMSLRSLKWSLLLLSLLSFFVMWYLSLPHYNVIERVNWM
YFYEYEPIYRQDFHFTLREHSNCSHQNPFLVILVTSHPSDVKARQAIRVT
WGEKKSWWGYEVLTFFLLGQEAEKEDKMLALSLEDEHLLYGDIIRQDFLD
TYNNLTLKTIMAFRWVTEFCPNAKYVMKTDTDVFINTGNLVKYLLNLNHS
EKFFTGYPLIDNYSYRGFYQKTHISYQEYPFKVFPPYCSGLGYIMSRDLV
PRIYEMMGHVKPIKFEDVYVGICLNLLKVNIHIPEDTNLFFLYRIHLDVC
QLRRVIAAHGFSSKEIITFWQVMLRNTTCHY

Exemplary cells that express P antigen include hematopoietic stem cells, megakaryocytes, endothelial cells, cardiomyocytes, hepatocytes, and trophoblasts. In some embodiments, the cell is a hematopoietic stem cell.

In some embodiments, an rAAV particle comprising an rAAV capsid protein as described herein, e.g., comprising one or more amino acid substitutions in a surface-exposed binding loop, is administered to a subject, e.g., to treat a disease, such as a hemoglobinopathy. In some embodiments, the method comprises administering a rAAV particle described herein to a subject (e.g., a human subject) having a hemoglobinopathy (e.g., β-thalassemia or sickle cell disease).

Yet other aspects of the disclosure relate to a method for efficient AAV transduction of a host cell suspension. In some embodiments, the method comprises contacting a host cell suspension with a recombinant AAV (rAAV) particle composition, wherein the host cell suspension has a density of greater than 4,000 cells per microliter (e.g., greater than 4,000 cells per microliter, greater than 5,000 cells per microliter, greater than 6,000 cells per microliter, greater than 7,000 cells per microliter, greater than 8,000 cells per microliter, greater than 9,000 cells per microliter, greater than 10,000 cells per microliter, greater than 11,000 cells per microliter, greater than 12,000 cells per microliter, greater than 13,000 cells per microliter, greater than 14,000 cells per microliter, or greater than 15,000 cells per microliter). In some embodiments, the host cell suspension has a density of 4,000 cells per microliter to 15,000 cells per microliter.

In some embodiments, a host cell suspension is a culture of cells that are in suspension (e.g., containing less than 10%, less than 5%, or less than 1% cells that are adhered to a solid substrate). The host cell suspension may contain non-adherent host cells or adherent host cells that have been treated such that they are no longer adherent (e.g., treated with trypsin or another protease or other molecule that disrupts adherence). In some embodiments, the host cell suspension is a non-adherent host cell suspension. In some embodiments, the non-adherent host cell is a human cell, such as a human stem cell. In some embodiments, the non-adherent host cell is a hematopoietic stem cell. In some embodiments, the host cell is obtained from a subject as described herein (e.g., is a primary cell). In some embodiments, the host cell is obtained from a cell line.

In some embodiments, the host cell suspension comprises culture medium, such as serum-free culture medium. Exemplary culture media includes Dulbecco's Modified Eagle Medium (DMEM), RPMI 1640, F10 Nutrient Mixture, Ham's F12 Nutrient Mixture, and Minimum Essential Media, all of which are known in the art and commercially available (see, e.g., products available from Life Technologies).

In some embodiments, an rAAV particle composition contacted with a host cell suspension contains 1×10²-1×10⁶, 1×10³-1×10⁶, 1×10³-1×10⁵, or 1×10³-1×10⁴ vector genomes(vgs)/mL of rAAV particles.

In some embodiments, host cells that have been transduced with an rAAV particle composition, e.g., by a method described herein, are administered to a subject. In some embodiments, the host cells are obtained from the subject, transduced with the rAAV particle composition, and then administered to the subject.

Other aspects of the disclosure relate to a method of treating a hemoglobinopathy, e.g., by administering host cells produced by a method described herein. In some embodiments, the method comprises administering an rAAV particle composition described herein to a host cell of a subject (e.g., a human subject) having a hemoglobinopathy (e.g., β-thalassemia or sickle cell disease) and subsequently administering the host cell to the subject.

Other aspects of the disclosure relate to a method of treating a disease involving blood cells, e.g., by administering host cells produced by a method described herein. In some embodiments, the method comprises administering an rAAV particle composition described herein to a host cell of a subject (e.g., a human subject) having the disease and subsequently administering the host cell to the subject. Exemplary blood cells include T cell, B cells, dendritic cells, macrophages, monocytes, and hematopoietic stem cells. In some embodiments, the disease is a blood cell cancer, e.g., a leukemia (such as Acute lymphocytic leukemia, Acute myelogenous leukemia, Chronic lymphocytic leukemia, or Chronic myelogenous leukemia), lymphoma (such as Hodgkin lymphoma or non-Hodgkin lymphoma), or myeloma (such as multiple myeloma). Other exemplary diseases involving blood cells include anemia, hemophilia, myelodysplastic syndrome, sickle cell disease, thalassemia, deep vein thrombosis, von Willebrand disease, factor II, V, VII, X, or XII deficiency, Polycythemia vera, thrombocytopenia and Idiopathic thrombocytopenic purpura.

Other aspects of the disclosure relate to a method of treating cancer, e.g., by administering host cells produced by a method described herein. In some embodiments, the method comprises administering an rAAV particle composition described herein to a host cell of a subject (e.g., a human subject) having cancer and subsequently administering the host cell to the subject. Exemplary cancers include breast cancer, prostate cancer, ovarian cancer, cervical cancer, skin cancer, pancreatic cancer, colorectal cancer, renal cancer, liver cancer, brain cancer, lymphoma, leukemia, myeloma, lung cancer and the like.

The rAAV particle, nucleic acid vector, or host cell may be delivered in the form of a composition, such as a composition comprising the active ingredient, such as a rAAV particle, nucleic acid, or host cell described herein, and a therapeutically or pharmaceutically acceptable carrier. The rAAV particles, nucleic acid vectors, or host cells may be prepared in a variety of compositions, and may also be formulated in appropriate pharmaceutical vehicles for administration to human or animal subjects.

The disclosure also provides compositions comprising one or more of the disclosed nucleic acid vectors, rAAV particles, or host cells. As described herein, such compositions may further comprise a pharmaceutical excipient, buffer, or diluent, and may be formulated for administration to an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

In some embodiments, the number of rAAV particles administered to a cell or a subject may be on the order ranging from 10⁶ to 10¹⁴ particles/mL or 10³ to 10¹⁵ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vector genomes(vgs)/mL or 10³ to 10¹⁵ vgs/mL, or any values therebetween for either range, such as for example, about 10⁶, 10⁷, 10⁸, 10⁹, 10¹⁰, 10¹¹, 10¹², 10¹³, or 10¹⁴ vgs/mL. In one embodiment, rAAV particles of higher than 10¹³ vgs/mL are be administered. The rAAV particles can be administered as a single dose, or divided into two or more administrations as may be required to achieve therapy of the particular disease or disorder being treated. In some embodiments, 0.0001 mL to 10 mLs are delivered to a subject.

In some embodiments, the disclosure provides formulations of one or more rAAV-based compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or host cell) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver an rAAV particle or host cell in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the rAAV particle or host cell compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle or host cell is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or host cells in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells (e.g., host cells) transduced with rAAV particles is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the disclosure.

The amount of rAAV particle, nucleic acid vector, or host cell compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or host cell compositions, either over a relatively short, or a relatively prolonged period of time, as may be determined by the medical practitioner overseeing the administration of such compositions.

The composition may include rAAV particles or host cells, either alone, or in combination with one or more additional active ingredients, which may be obtained from natural or recombinant sources or chemically synthesized. In some embodiments, rAAV particles are administered in combination, either in the same composition or administered as part of the same treatment regimen, with a proteasome inhibitor, such as Bortezomib, or hydroxyurea.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Recombinant AAV (rAAV) Particles and Nucleic Acid Vectors

Aspects of the disclosure relate to recombinant adeno-associated virus (rAAV) particles for delivery of one or more nucleic acid vectors comprising a sequence encoding a protein or polypeptide of interest into various tissues, organs, and/or cells. In some embodiments, the rAAV particles comprise a rAAV capsid protein as described herein, e.g., comprising one or more amino acid substitutions in a surface-exposed binding loop.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector, which may comprise at a minimum (a) one or more heterologous nucleic acid regions comprising a sequence encoding a protein or polypeptide of interest (e.g., a globin gene) or an RNA of interest (e.g., a siRNA or microRNA) and (b) one or more regions comprising inverted terminal repeat (ITR) sequences (e.g., wild-type ITR sequences or engineered ITR sequences) flanking the one or more heterologous nucleic acid regions. In some embodiments, the nucleic acid vector is between 4 kb and 5 kb in size (e.g., 4.2 to 4.7 kb in size). This nucleic acid vector may be encapsidated by a viral capsid, such as an AAV2 or AAV6 capsid, which may comprise a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Accordingly, in some embodiments, an rAAV particle comprises a viral capsid and a nucleic acid vector as described herein, which is encapsidated by the viral capsid. In some embodiments, the nucleic acid vector comprises (1) one or more heterologous nucleic acid regions comprising a sequence encoding a protein or polypeptide of interest (e.g., a globin gene), (2) one or more nucleic acid regions comprising a sequence that facilitates expression of the heterologous nucleic acid region (e.g., a parvovirus B19p6 promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more heterologous nucleic acid regions comprising a sequence encoding a protein or polypeptide of interest operably linked to a promoter, wherein the one or more heterologous nucleic acid regions are flanked on each side with an ITR sequence. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™ Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

Exemplary ITR sequences for AAV2, AAV3, AAV5, and AAV6 are provided below.

AAV2:
(SEQ ID NO: 3)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT
AAV3:
(SEQ ID NO: 4)
TTGGCCACTCCCTCTATGCGCACTCGCTCGCTCGGTGGGGCCTGGCGACC
AAAGGTCGCCAGACGGACGTGCTTTGCACGTCCGGCCCCACCGAGCGAGC
GAGTGCGCATAGAGGGAGTGGCCAACTCCATCACTAGAGGTATGGC
AAV5:
(SEQ ID NO: 5)
CTCTCCCCCCTGTCGCGTTCGCTCGCTCGCTGGCTCGTTTGGGGGGGTGG
CAGCTCAAAGAGCTGCCAGACGACGGCCCTCTGGCCGTCGCCCCCCCAAA
CGAGCCAGCGAGCGAGCGAACGCGACAGGGGGGAGAGTGCCACACTCTCA
AGCAAGGGGGTTTTGTA
AAV6:
(SEQ ID NO: 6)
TTGCCCACTCCCTCTATGCGCGCTCGCTCGCTCGGTGGGGCCTGCGGACC
AAAGGTCCGCAGACGGCAGAGCTCTGCTCTGCCGGCCCCACCGAGCGAGC
GAGCGCGCATAGAGGGAGTGGGCAACTCCATCACTAGGGGTA

In some embodiments, the nucleic acid vector comprises one or more regions comprising a sequence that facilitates expression of the heterologous nucleic acid, e.g., expression control sequences operatively linked to the heterologous nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. In some embodiments, the promoter is a parvovirus B19p6 promoter. An exemplary sequence of the parvovirus B19p6 promoter is provided below:

(SEQ ID NO: 7)

| | |
|---|---|
| 1 | CCAACCCTAA TTCCGGAAGT CCCGCCCACC GGAAGTGACG |
| | TCACAGGAAA TGACGTCACA |
| 61 | GGAAATGACG TAATTGTCCG CCATCTTGTA CCGGAAGTCC |
| | CGCCTACCGG CGGCGACCGG |
| 121 | CGGCATCTGA TTTGGTGTCT TCTTTTAAAT TTTAGCGGGC |
| | TTTTTTCCCG CCTTATGCAA |
| 181 | ATGGGCAGCC ATTTTAAGTG TTTTACTATA ATTTTATTGG |
| | TTAGTTTTGT AACGGTTAAA |
| 241 | ATGGGCGGAG CGTAGGCGGG GACTACAGTA TATATAGCAC |
| | GGCACTGCCG CAGCTCTTTC |
| 301 | TTTCTGGGCT GCTTTTTCCT GGACTTTCTT GCTGTTTTTT |
| | GTGAGCTAAC TAACAGGTAT |
| 361 | TTATACTACT TGTTAACATA CTAA |

The promoter may be, for example, a constitutive promoter, tissue-specific promoter, inducible promoter, or a synthetic promoter. For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include the parvovirus B19p6 promoter, promoters that are myeloid and erythroid cell-specific, dendritic cell-specific, macrophage- and monocyte-specific, T- and B-lymphocyte-specific, specific for hematopoietic stem or progenitor cells, dendritic cells, macrophages or monocytes.

Synthetic promoters are also contemplated herein. A synthetic promoter may comprise, for example, regions of known promoters, regulatory elements, transcription factor binding sites, enhancer elements, repressor elements, and the like.

In some embodiments, a nucleic acid vector described herein may also contain marker or reporter genes, e.g., LacZ or a fluorescent protein.

In some embodiments, the nucleic acid vector comprises one or more heterologous nucleic acid regions comprising a sequence encoding a protein or polypeptide of interest, such as a globin gene. Exemplary globin genes include, but are not limited to, a β-globin gene (e.g., a human β-globin gene), an anti-sickling β-globin gene (e.g., a human anti-sickling 3-globin gene), and a γ-globin gene (e.g., a human γ-globin gene). Exemplary nucleic acid and protein sequences for each globin gene mentioned above are provided below.

### Human β-Globin Protein:

(SEQ ID NO: 8)
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

### Human γ-Globin Protein:

(SEQ ID NO: 9)
MGHFTEEDKATITSLWGKVNVEDAGGETLGRLLVVYPWTQRFFDSFG
NLSSASAIMGNPKVKAHGKKVLTSLGDAIKHLDDLKGTFAQLSELHC
DKLHVDPENFKLLGNVLVTVLAIHFGKEFTPEVQASWQKMVTGVASA
LSSRYH

### Human Anti-Sickling β-Globin Gene Nucleic Acid Sequence:

Exon 1
(SEQ ID NO: 10)
ATGGTGCACCTGACTCCTGAGGAGAAGTCTGCCGTTACTGCCCTGTGGGG
CAAGGTGAACGTGGATGAAGTTGGTGGTGAGGCCCTGGGCA
Intron 1
(SEQ ID NO: 11)
GGTTGGTATCAAGGTTACAAGACAGGTTTAAGGAGACCAATAGAAACTGG
GCATGTGGAGACAGAGAAGACTCTTGGGTTTCTGATAGGCACTGACTCTC
TCTGCCTATTGGTCTATTTTCCCACCCTTA
Exon 2
(SEQ ID NO: 12)
GGCTGCTGGTGGTCTACCCTTGGACCCAGAGGTTCTTTGAGTCCTTTGGG
GATCTGTCCACTCCTGATGCTGTTATGGGCAACCCTAAGGTGAAGGCTCA
TGGCAAGAAAGTGCTCGGTGCCTTTAGTGATGGCCTGGCTCACCTGGACA
ACCTCAAGGGCACCTTTGCC*CAG*CTGAGTGAGCTGCACTGTGACAAGC
TGCACGTGGATCCTGAGAACTTCAGG
Delta 12 Intron (372 bp-deletion in Intron 2)
(SEQ ID NO: 13)
GTGAGTCTATGGGACCCTTGATGTTTTCTTTCCCCTTCTTTTCTATGGTT
AAGTTCATGTCATAGGAAGGGGAGAAGTAACAGGGTACACATATTGACCA
AATCAGGGTAATTTTGCATTTGTAATTTTAAAAAATGCTTTCTTCTTTTA
ATATACTTTTTTGTTTATCTTATTTCTAATACTTTCCCTAATCTCTTTCT
TTCAGGGCAATAATGATACAATGTATCATGCCTCTTTGCACCATTCTAAA
GAATAACAGTGATAATTTCTGGGTTAAGGCAATAGCAATATTTCTGCATA
TAAATATTTCTGCATATAAATTGTAACTGATGTAAGAGGTTTCATATTGC
TAATAGCAGCTACAATCCAGCTACCATTCTGCTTTTATTTTATGGTTGGG
ATAAGGCTGGATTATTCTGAGTCCAAGCTAGGCCCTTTTGCTAATCATGT
TCATACCTCTTATCTTCCTCCCACAG
Exon 3
(SEQ ID NO: 14)
CTCCTGGGCAACGTGCTGGTCTGTGTGCTGGCCCATCACTTTGGCAAAGA
ATTCACCCCACCAGTGCAGGCTGCCTATCAGAAAGTGGTGGCTGGTGTGG
CTAATGCCCTGGCCCACAAGTATCACTAA
*CAG* = T87Q

In some embodiments, the sequence encoding the globin gene is provided with introns. In some embodiments, the sequence encoding the globin gene is provided without introns.

### Human Anti-Sickling β-Globin Gene Protein Sequence:

(SEQ ID NO: 15)
MVHLTPEEKSAVTALWGKVNVDEVGGEALGRLLVVYPWTQRFFESFGDLS
TPDAVMGNPKVKAHGKKVLGAFSDGLAHLDNLKGTFAQLSELHCDKLHVD
PENFRLLGNVLVCVLAHHFGKEFTPPVQAAYQKVVAGVANALAHKYH

The protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications provided in Table 1

TABLE 1
Non-limiting examples of proteins or polypeptides
of interest and associated diseases

| | | Non-limiting NCBI |
|---|---|---|
| Protein or | Non-limiting | Protein IDs or |
| Polypeptide | Exemplary diseases | Patent SEQ ID NOs |
| acid alpha- | Pompe | NP_000143.2, |
| glucosidase (GAA) | | NP_001073271.1, |
| | | NP_001073272.1 |
| Methyl CpG binding | Rett syndrome | NP_001104262.1, |
| protein 2 (MECP2) | | NP_004983.1 |
| Aromatic L-amino | Parkinson's | NP_000781.1, |
| acid decarboxylase | disease | NP_001076440.1, |
| (AADC) | | NP_001229815.1, |
| | | NP_001229816.1, |
| | | NP_001229817.1, |
| | | NP_001229818.1, |
| | | NP_001229819.1 |
| Glial cell-derived | Parkinson's | NP_000505.1, |
| neurotrophic factor | disease | NP_001177397.1, |
| (GDNF) | | NP_001177398.1, |
| | | NP_001265027.1, |
| | | NP_954701.1 |
| Cystic fibrosis | Cystic fibrosis | NP_000483.3 |
| transmembrane | | |
| conductance regulator | | |
| (CFTR) | | |
| Tumor necrosis factor | Arthritis, | SEQ ID NO. 1 of |
| receptor fused | Rheumatoid | WO2013025079 |
| to an antibody Fc | arthritis | |
| (TNFR:Fc) | | |
| HIV-1 gag-proΔrt | HIV infection | SEQ ID NOs. 1-5 of |
| (tgAAC09) | | WO2006073496 |
| Sarcoglycan alpha, | Muscular | SGCA |
| beta, gamma, delta, | dystrophy | NP_000014.1, |
| epsilon, or zeta | | NP_001129169.1 |
| (SGCA, SGCB, | | SGCB |
| SGCG, SGCD, | | NP_000223.1 |
| SGCE, or SGCZ) | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Alpha-1-antitrypsin | Hereditary | NP_000286.3, |
| (AAT) | emphysema or | NP_001002235.1, |
| | Alpha-1- | NP_001002236.1, |
| | antitrypsin | NP_001121172.1, |
| | deficiency | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Glutamate | Parkinson's | NP_000808.2, |
| decarboxylase | disease | NP_038473.2 |
| 1(GAD1) | | |
| Glutamate | Parkinson's | NP_000809.1, |
| decarboxylase | disease | NP_001127838.1 |
| 2 (GAD2) | | |
| Aspartoacylase | Canavan's | NP_000040.1, |
| (ASPA) | disease | NP_001121557.1 |
| Nerve growth | Alzheimer's | NP_002497.2 |
| factor (NGF) | disease | |
| Granulocyte-macrophage | Prostate | NP_000749.2 |
| colonystimulating | cancer | |
| factory (GM-CSF) | | |
| Cluster of | Malignant | NP_001193853.1, |
| Differentiation | melanoma | NP_001193854.1, |
| 86 (CD86 or | | NP_008820.3, |
| B7-2) | | NP_787058.4, |
| | | NP_795711.1 |
| Interleukin 12 | Malignant | NP_000873.2, |
| (IL-12) | melanoma | NP_002178.2 |
| neuropeptide | Parkinson's | NP_000896.1 |
| Y (NPY) | disease, | |
| | epilepsy | |
| ATPase, Ca++ | Chronic heart | NP_001672.1, |
| transporting, cardiac | failure | NP_733765.1 |
| muscle, slow twitch | | |
| 2 (SERCA2) | | |
| Dystrophin or | Muscular | NP_000100.2, |
| Minidystrophin | dystrophy | NP_003997.1, |
| | | NP_004000.1, |
| | | NP_004001.1, |
| | | NP_004002.2, |
| | | NP_004003.1, |
| | | NP_004004.1, |
| | | NP_004005.1, |
| | | NP_004006.1, |
| | | NP_004007.1, |
| | | NP_004008.1, |
| | | NP_004009.1, |
| | | NP_004010.1, |
| | | NP_004011.2, |
| | | NP_004012.1, |
| | | NP_004013.1, |
| | | NP_004014.1 |
| Ceroid | Late infantile | NP_000382.3 |
| lipofuscinosis | neuronal | |
| neuronal | ceroidlipo- | |
| 2 (CLN2) | fuscinosis or | |
| | Batten's | |
| | disease | |
| Neurturin | Parkinson's | NP_004549.1 |
| (NRTN) | disease | |
| N-acetylglucos- | Sanfilippo | NP_000254.2 |
| aminidase, alpha | syndrome | |
| (NAGLU) | (MPSIIIB) | |
| Iduronidase, | MPSI-Hurler | NP_000194.2 |
| alpha-l (IDUA) | | |
| Iduronate | MPSII- | NP_000193.1, |
| 2-sulfatase | Hunter | NP_001160022.1, |
| (IDS) | | NP_006114.1 |
| Glucuronidase, | MPSVII-Sly | NP_000172.2, |
| beta (GUSB) | | NP_001271219.1 |
| Hexosaminidase A, | Tay-Sachs | NP_000511.2 |
| α polypeptide | | |
| (HEXA) | | |
| Retinal pigment | Leber | NP_000320.1 |
| epithelium-specific | congenital | |
| protein 65 kDa | amaurosis | |
| (RPE65) | | |
| Factor IX (FIX) | Hemophilia B | NP_000124.1 |
| Adenine | progressive | NP_001142.2 |
| nucleotide | external | |
| translocator | ophthalmoplegia | |
| (ANT-1) | | |
| ApaLI | mitochondrial | YP_007161330.1 |
| | heteroplasmy, | |
| | myoclonic epilepsy | |
| | with ragged red | |
| | fibers (MERRF) or | |
| | mitochondrial | |
| | encephalomyopathy, | |
| | lactic acidosis, | |
| | and stroke-like | |
| | episodes (MELAS) | |
| NADH ubiquinone | Leber hereditary | YP_003024035.1 |
| oxidoreductase | optic | |
| subunit 4 (ND4) | | |
| very long- | very long-chain | NP_000009.1, |
| acyl-CoA | acyl-CoA | NP_001029031.1, |
| dehydrogenase | dehydrogenase | NP_001257376.1, |
| (VLCAD) | (VLCAD) | NP_001257377.1 |
| | deficiency | |
| short-chain | short-chain | NP_000008.1 |
| acyl-CoA | acyl-CoA | |
| dehydrogenase | dehydrogenase | |
| (SCAD) | (SCAD) | |
| | deficiency | |
| medium-chain | medium-chain | NP_000007.1, |
| acyl-CoA | acyl-CoA | NP_001120800.1, |
| dehydrogenase | dehydrogenase | NP_001272971.1, |
| (MCAD) | (MCAD) | NP_001272972.1, |
| | deficiency | NP_001272973.1 |
| Myotubularin 1 | X-linked | NP_000243.1 |
| (MTM1) | myotubular | |
| | myopathy | |
| Myophosphorylase | McArdle disease | NP_001158188.1, |
| (PYGM) | (glycogen | NP_005600.1 |
| | storage disease | |
| | type V, | |
| | myophosphorylase | |
| | deficiency) | |
| Lipoprotein lipase | LPL deficiency | NP_000228.1 |
| (LPL) | | |
| sFLT01 (VEGF/PlGF | Age-related | SEQ ID NO: 2, 8, |
| (placental growth | macular | 21, 23, or 25 of |
| factor) binding | degeneration | WO2009105669 |
| domain of human | | |
| VEGFR1/Flt-1 | | |
| (hVEGFR1) fused to | | |
| the Fc portion of | | |
| human IgG(1) through | | |
| a polyglycine linker) | | |
| Glucocerebrosidase | Gaucher | NP_000148.2, |
| (GC) | disease | NP_001005741.1, |
| | | NP_001005742.1, |
| | | NP_001165282.1, |
| | | NP_001165283.1 |
| UDP glucuronosyl- | Crigler-Najjar | NP_000454.1 |
| transferase 1 | syndrome | |
| family, polypep- | | |
| tide A1 (UGT1A1) | | |
| Glucose 6-phosphatase | GSD-Ia | NP_000142.2, |
| (G6Pase) | | NP_001257326.1 |
| Ornithine carbamoyl- | OTC | NP_000522.3 |
| transferase (OTC) | deficiency | |
| Cystathionine- | Homocystinuria | NP_000062.1, |
| beta-synthase | | NP_001171479.1, |
| (CBS) | | NP_001171480.1 |
| Factor VIII | Haemophilia | NP_000123.1, |
| (F8) | A | NP_063916.1 |
| Hemochromatosis | Hemochromatosis | NP_000401.1, |
| (HFE) | | NP_620572.1, |
| | | NP_620573.1, |
| | | NP_620575.1, |
| | | NP_620576.1, |
| | | NP_620577.1, |
| | | NP_620578.1, |
| | | NP_620579.1, |
| | | NP_620580.1 |
| Low density | Phenylketonuria | NP_000518.1, |
| lipoprotein | (PKU) | NP_001182727.1, |
| receptor | | NP_001182728.1, |
| (LDLR) | | NP_001182729.1, |
| | | NP_001182732.1 |
| Galactosidase, | Fabry disease | NP_000160.1 |
| alpha (AGA) | | |
| Phenylalanine | Hypercholes- | NP_000268.1 |
| hydroxylase | terolaemia or | |
| (PAH) | Phenylketonuria | |
| | (PKU) | |
| Propionyl CoA | Propionic | NP_000273.2, |
| carboxylase, | acidaemias | NP_001121164.1, |
| alpha polypeptide | | NP_001171475.1 |
| (PCCA) | | |

Other exemplary polypeptides or proteins of interest include adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphorin a semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

The rAAV particle may be of any AAV serotype, including any derivative or pseudotype (e.g., 1, 2, 3, 4, 5, 6, 7, 8, 9, 10, 2/1, 2/5, 2/8, or 2/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is AAV2. In some embodiments, the rAAV particle is AAV6. In some embodiments, the rAAV particle is an AAV6 serotype comprising an rAAV capsid protein as described herein. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAV5hH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some embodiments, the rAAV particle comprises a capsid that includes modified capsid proteins (e.g., capsid proteins comprising a modified VP3 region and/or one or more amino acid substitutions in a surface exposed loop, such as by replacing loop VIII with a B19 P antigen binding site) optionally further modified to replace one or more surface exposed tyrosine, lysine, serine, or threonine residues (e.g., in a VP3 region of a capsid protein, see, e.g., U.S Patent Publication Number US20130310443, which is incorporated herein by reference in its entirety). In some embodiments, the rAAV particle comprises a modified capsid protein comprising a non-tyrosine residue (e.g., a phenylalanine) at a position that corresponds to a surface-exposed tyrosine residue in a wild-type capsid protein, a non-threonine residue (e.g., a valine) at a position that corresponds to a surface-exposed threonine residue in the wild-type capsid protein, a non-lysine residue (e.g., a glutamic acid) at a position that corresponds to a surface-exposed lysine residue in the wild-type capsid protein, a non-serine residue (e.g., valine) at a position that corresponds to a surface-exposed serine residue in the wild-type capsid protein, or a combination thereof. Exemplary surface-exposed lysine residues include positions that correspond to K258, K321, K459, K490, K507, K527, K572, K532, K544, K549, K556, K649, K655, K665, or K706 of the wild-type AAV2 capsid protein. Exemplary surface-exposed serine residues include positions that correspond to S261, 5264, 5267, 5276, 5384, 5458, 5468, 5492, 5498, 5578, 5658, 5662, 5668, 5707, or S721 of the wild-type AAV2 capsid protein. Exemplary surface-exposed threonine residues include positions that correspond to T251, T329, T330, T454, T455, T503, T550, T592, T581, T597, T491, T671, T659, T660, T701, T713, or T716 of the wild-type AAV2 capsid protein. Exemplary surface-exposed tyrosine residues include positions that correspond to Y252, Y272, Y444, Y500, Y700, Y704, or Y730 of the wild-type AAV2 capsid protein.

Exemplary, non-limiting wild-type capsid protein sequences are provided below.

### Exemplary AAV1 Capsid Protein

(SEQ ID NO: 16)

| | |
|---|---|
| 1 | MAADGYLPDW LEDNLSEGIR EWWDLKPGAP KPKANQQKQD |
| | DGRGLVLPGY |
| 51 | KYLGPFNGLD KGEPVNAADA AALEHDKAYD QQLKAGDNPY |
| | LRYNHADAEF |
| 101 | QERLQEDTSF GGNLGRAVFQ AKKRVLEPLG LVEEGAKTAP |
| | GKKRPVEQSP |
| 151 | QEPDSSSGIG KTGQQPAKKR LNFGQTGDSE SVPDPQPLGE |
| | PPATPAAVGP |
| 201 | TTMASGGGAP MADNNEGADG VGNASGNWHC DSTWLGDRVI |
| | TTSTRTWALP |
| 251 | TYNNHLYKQI SSASTGASND NHYFGYSTPW GYFDFNRFHC |
| | HFSPRDWQRL |
| 301 | INNNWGFRPK RLNFKLFNIQ VKEVTTNDGV TTIANNLTST |
| | VQVFSDSEYQ |
| 351 | LPYVLGSAHQ GCLPPFPADV FMIPQYGYLT LNNGSQAVGR |
| | SSFYCLEYFP |
| 401 | SQMLRTGNNF TFSYTFEEVP FHSSYAHSQS LDRLMNPLID |
| | QYLYYLNRTQ |
| 451 | NQSGSAQNKD LLFSRGSPAG MSVQPKNWLP GPCYRQQRVS |
| | KTKTDNNNSN |
| 501 | FTWTGASKYN LNGRESIINP GTAMASHKDD EDKFFPMSGV |
| | MIFGKESAGA |
| 551 | SNTALDNVMI TDEEEIKATN PVATERFGTV AVNFQSSSTD |
| | PATGDVHAMG |
| 601 | ALPGMVWQDR DVYLQGPIWA KIPHTDGHFH PSPLMGGFGL |
| | KNPPPQILIK |
| 651 | NTPVPANPPA EFSATKFASF ITQYSTGQVS VEIEWELQKE |
| | NSKRWNPEVQ |
| 701 | YTSNYAKSAN VDFTVDNNGL YTEPRPIGTR YLTRPL* |

### Exemplary AAV2 Capsid Protein

(SEQ ID NO: 17)

| | |
|---|---|
| 1 | MAADGYLPDW LEDTLSEGIR QWWKLKPGPP PPKPAERHKD |
| | DSRGLVLPGY |
| 51 | KYLGPFNGLD KGEPVNEADA AALEHDKAYD RQLDSGDNPY |
| | LKYNHADAEF |
| 101 | QERLKEDTSF GGNLGRAVFQ AKKRVLEPLG LVEEPVKTAP |
| | GKKRPVEHSP |
| 151 | VEPDSSSGTG KAGQQPARKR LNFGQTGDAD SVPDPQPLGQ |
| | PPAAPSGLGT |
| 201 | NTMATGSGAP MADNNEGADG VGNSSGNWHC DSTWMGDRVI |
| | TTSTRTWALP |
| 251 | TYNNHLYKQI SSQSGASNDN HYFGYSTPWG YFDFNRFHCH |
| | FSPRDWQRLI |
| 301 | NNNWGFRPKR LNFKLFNIQV KEVTQNDGTT TIANNLTSTV |
| | QVFTDSEYQL |
| 351 | PYVLGSAHQG CLPPFPADVF MVPQYGYLTL NNGSQAVGRS |
| | SFYCLEYFPS |
| 401 | QMLRTGNNFT FSYTFEDVPF HSSYAHSQSL DRLMNPLIDQ |
| | YLYYLSRTNT |
| 451 | PSGTTTQSRL QFSQAGASDI RDQSRNWLPG PCYRQQRVSK |
| | TSADNNNSEY |
| 501 | SWTGATKYHL NGRDSLVNPG PAMASHKDDE EKFFPQSGVL |
| | IFGKQGSEKT |
| 551 | NVDIEKVMIT DEEEIRTTNP VATEQYGSVS TNLQRGNRQA |
| | ATADVNTQGV |
| 601 | LPGMVWQDRD VYLQGPIWAK IPHTDGHFHP SPLMGGFGLK |
| | HPPPQILIKN |
| 651 | TPVPANPSTT FSAAKFASFI TQYSTGQVSV EIEWELQKEN |
| | SKRWNPEIQY |
| 701 | TSNYNKSVNV DFTVDTNGVY SEPRPIGTRY LTRNL* |

### Exemplary AAV3 Capsid Protein

| (SEQ ID NO: 18) | |
|---|---|
| 1 | MAADGYLPDW LEDNLSEGIR EWWALKPGVP QPKANQQHQD NRRGLVLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPF HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTTN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDTNGV YSEPRPIGTR YLTRNL* |

### Exemplary AAV4 Capsid Protein

| (SEQ ID NO: 19) | |
|---|---|
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | QRLQGDTSFG GNLGRAVFQA KKRVLEPLGL VEQAGETAPG KKRPLIESPQ |
| 151 | QPDSSTGIGK KGKQPAKKKL VFEDETGAGD GPPEGSTSGA MSDDSEMRAA |
| 201 | AGGAAVEGGQ GADGVGNASG DWHCDSTWSE GHVTTTSTRT WVLPTYNNHL |
| 251 | YKRLGESLQS NTYNGFSTPW GYFDFNRFHC HFSPRDWQRL INNNWGMRPK |
| 301 | AMRVKIFNIQ VKEVTTSNGE TTVANNLTST VQIFADSSYE LPYVMDAGQE |
| 351 | GSLPPFPNDV FMVPQYGYCG LVTGNTSQQQ TDRNAFYCLE YFPSQMLRTG |
| 401 | NNFEITYSFE KVPFHSMYAH SQSLDRLMNP LIDQYLWGLQ STTTGTTLNA |
| 451 | GTATTNFTKL RPTNFSNFKK NWLPGPSIKQ QGFSKTANQN YKIPATGSDS |
| 501 | LIKYETHSTL DGRWSALTPG PPMATAGPAD SKFSNSQLIF AGPKQNGNTA |
| 551 | TVPGTLIFTS EEELAATNAT DTDMWGNLPG GDQSNSNLPT VDRLTALGAV |
| 601 | PGMVWQNRDI YYQGPIWAKI PHTDGHFHPS PLIGGFGLKH PPPQIFIKNT |
| 651 | PVPANPATTF SSTPVNSFIT QYSTGQVSVQ IDWEIQKERS KRWNPEVQFT |
| 701 | SNYGQQNSLL WAPDAAGKYT EPRAIGTRYL THHL* |

### Exemplary AAV5 Capsid Protein

| (SEQ ID NO: 20) | |
|---|---|
| 1 | MSFVDHPPDW LEEVGEGLRE FLGLEAGPPK PKPNQQHQDQ ARGLVLPGYN |
| 51 | YLGPGNGLDR GEPVNRADEV AREHDISYNE QLEAGDNPYL KYNHADAEFQ |
| 101 | EKLADDTSFG GNLGKAVFQA KKRVLEPFGL VEEGAKTAPT GKRIDDHFPK |
| 151 | RKKARTEEDS KPSTSSDAEA GPSGSQQLQI PAQPASSLGA DTMSAGGGGP |
| 201 | LGDNNQGADG VGNASGDWHC DSTWMGDRVV TKSTRTWVLP SYNNHQYREI |
| 251 | KSGSVDGSNA NAYFGYSTPW GYFDFNRFHS HWSPRDWQRL INNYWGFRPR |
| 301 | SLRVKIFNIQ VKEVTVQDST TTIANNLTST VQVFTDDDYQ LPYVVGNGTE |
| 351 | GCLPAFPPQV FTLPQYGYAT LNRDNTENPT ERSSFFCLEY FPSKMLRTGN |
| 401 | NFEFTYNFEE VPFHSSFAPS QNLFKLANPL VDQYLYRFVS TNNTGGVQFN |
| 451 | KNLAGRYANT YKNWFPGPMG RTQGWNLGSG VNRASVSAFA TTNRMELEGA |
| 501 | SYQVPPQPNG MTNNLQGSNT YALENTMIFN SQPANPGTTA TYLEGNMLIT |
| 551 | SESETQPVNR VAYNVGGQMA TNNQSSTTAP ATGTYNLQEI VPGSVWMERD |
| 601 | VYLQGPIWAK IPETGAHFHP SPAMGGFGLK HPPPMMLIKN TPVPGNITSF |
| 651 | SDVPVSSFIT QYSTGQVTVE MEWELKKENS KRWNPEIQYT NNYNDPQFVD |
| 701 | FAPDSTGEYR TTRPIGTRYL TRPL* |

### Exemplary AAV6 Capsid Protein

| (SEQ ID NO: 21) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL* |

### Exemplary AAV7 Capsid Protein

| (SEQ ID NO: 22) | |
|---|---|
| 1 | MAADGYLPDW LEDNLSEGIR EWWDLKPGAP KPKANQQKQD NGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP AKKRPVEPSP |
| 151 | QRSPDSSTGI GKKGQQPARK RLNFGQTGDS ESVPDPQPLG EPPAAPSSVG |
| 201 | SGTVAAGGGA PMADNNEGAD GVGNASGNWH CDSTWLGDRV ITTSTRTWAL |
| 251 | PTYNNHLYKQ ISSETAGSTN DNTYFGYSTP WGYFDFNRFH CHFSPRDWQR |
| 301 | LINNNWGFRP KKLRFKLFNI QVKEVTTNDG VTTIANNLTS TIQVFSDSEY |
| 351 | QLPYVLGSAH QGCLPPFPAD VFMIPQYGYL TLNNGSQSVG RSSFYCLEYF |
| 401 | PSQMLRTGNN FEFSYSFEDV PFHSSYAHSQ SLDRLMNPLI DQYLYYLART |
| 451 | QSNPGGTAGN RELQFYQGGP STMAEQAKNW LPGPCFRQQR VSKTLDQNNN |
| 501 | SNFAWTGATK YHLNGRNSLV NPGVAMATHK DDEDRFFPSS GVLIFGKTGA |
| 551 | TNKTTLENVL MTNEEEIRPT NPVATEEYGI VSSNLQAANT AAQTQVVNNQ |
| 601 | GALPGMVWQN RDVYLQGPIW AKIPHTDGNF HPSPLMGGFG LKHPPPQILI |
| 651 | KNTPVPANPP EVFTPAKFAS FITQYSTGQV SVEIEWELQK ENSKRWNPEI |
| 701 | QYTSNFEKQT GVDFAVDSQG VYSEPRPIGT RYLTRNL* |

### Exemplary AAV8 Capsid Protein

| (SEQ ID NO: 23) | |
|---|---|
| 1 | MAADGYLPDW LEDNLSEGIR EWWALKPGAP KPKANQQKQD DGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPARK RLNFGQTGDS ESVPDPQPLG EPPAAPSGVG |
| 201 | PNTMAAGGGA PMADNNEGAD GVGSSSGNWH CDSTWLGDRV ITTSTRTWAL |
| 251 | PTYNNHLYKQ ISNGTSGGAT NDNTYFGYST PWGYFDFNRF HCHFSPRDWQ |
| 301 | RLINNNWGFR PKRLSFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFQFTYTFED VPFHSSYAHS QSLDRLMNPL IDQYLYYLSR |
| 451 | TQTTGGTANT QTLGFSQGGP NTMANQAKNW LPGPCYRQQR VSTTTGQNNN |
| 501 | SNFAWTAGTK YHLNGRNSLA NPGIAMATHK DDEERFFPSN GILIFGKQNA |
| 551 | ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL |
| 651 | IKNTPVPADP PTTFNQSKLN SFITQYSTGQ VSVEIEWELQ KENSKRWNPE |
| 701 | IQYTSNYYKS TSVDFAVNTE GVYSEPRPIG TRYLTRNL* |

### Exemplary AAV9 Capsid Protein

| (SEQ ID NO: 24) | |
|---|---|
| 1 | MAADGYLPDW LEDNLSEGIR EWWALKPGAP QPKANQQHQD NARGLVLPGY |
| 51 | KYLGPGNGLD KGEPVNAADA AALEHDKAYD QQLKAGDNPY LKYNHADAEF |
| 101 | QERLKEDTSF GGNLGRAVFQ AKKRLLEPLG LVEEAAKTAP GKKRPVEQSP |
| 151 | QEPDSSAGIG KSGAQPAKKR LNFGQTGDTE SVPDPQPIGE PPAAPSGVGS |
| 201 | LTMASGGGAP VADNNEGADG VGSSSGNWHC DSQWLGDRVI TTSTRTWALP |
| 251 | TYNNHLYKQI SNSTSGGSSN DNAYFGYSTP WGYFDFNRFH CHFSPRDWQR |
| 301 | LINNNWGFRP KRLNFKLFNI QVKEVTDNNG VKTIANNLTS TVQVFTDSDY |
| 351 | QLPYVLGSAH EGCLPPFPAD VFMIPQYGYL TLNDGSQAVG RSSFYCLEYF |
| 401 | PSQMLRTGNN FQFSYEFENV PFHSSYAHSQ SLDRLMNPLI DQYLYYLSKT |
| 451 | INGSGQNQQT LKFSVAGPSN MAVQGRNYIP GPSYRQQRVS TTVTQNNNSE |
| 501 | FAWPGASSWA LNGRNSLMNP GPAMASHKEG EDRFFPLSGS LIFGKQGTGR |
| 551 | DNVDADKVMI TNEEEIKTTN PVATESYGQV ATNHQSAQAQ AQTGWVQNQG |
| 601 | ILPGMVWQDR DVYLQGPIWA KIPHTDGNFH PSPLMGGFGM KHPPPQILIK |
| 651 | NTPVPADPPT AFNKDKLNSF ITQYSTGQVS VEIEWELQKE NSKRWNPEIQ |
| 701 | YTSNYYKSNN VEFAVNTEGV YSEPRPIGTR YLTRNL* |

### Exemplary AAV10 Capsid Protein

| (SEQ ID NO: 25) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYQFED VPFHSSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERFFPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL* |

In some embodiments, the modified capsid protein comprises a non-tyrosine residue and/or a non-threonine residue at one or more of or each of Y705, Y731, and T492 of a wild-type AAV6 capsid protein (see sequence below with Y705, Y731, and T492 positions underlined, bolded and italicized). In some embodiments, the non-tyrosine residue is phenylalanine and the non-threonine residue is valine.

| (SEQ ID NO: 21) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYFLNRTQ |
| 451 | NQSGSAQNKD LLFSRGSPAG MSVQPKNWLP GPCYRQQRVS K [symbol] KTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSN [symbol] AKSAN VDFTVDNNGL YTEPRPIGTR [symbol] LTRPL |

In some embodiments, the modified capsid protein comprises a non-tyrosine residue and/or a non-threonine residue at one or more of or each of Y444, Y500, Y731, and T491 of a wild-type AAV2 capsid protein (see sequence below with Y444, Y500, Y731, and T491 positions underlined, bolded and italicized). In some embodiments, the non-tyrosine residue is phenylalanine and the non-threonine residue is valine.

| (SEQ ID NO: 17) | |
|---|---|
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLY [symbol] LSRTNT |
| 451 | PSGTTTQSRL QFSQAGASDI RDQSRNWLPG PCYRQQRVSK [symbol] SADNNNSE [symbol] |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDTNGVY SEPRPIGTR [symbol] LTRNL |

Other aspects of the disclosure relate to the nucleic acid vector. In some embodiments, the nucleic acid vector is provided in a form suitable for inclusion in a rAAV particle, such as a single-stranded or self-complementary nucleic acid. In some embodiments, the nucleic acid vector is provided in a form suitable for use in a method of producing rAAV particles. For example, in some embodiments, the nucleic acid vector is a plasmid (e.g., comprising an origin of replication (such as an E. coli ORI) and optionally a selectable marker (such as an Ampicillin or Kanamycin selectable marker)). In some embodiments, the nucleic acid vector comprises a parvovirus B19p6 promoter operatively linked to a globin gene, wherein the promoter and gene are flanked by ITR sequences, such as AAV2 or AAV6 ITR sequences. In some embodiments, the nucleic acid vector comprises the sequence as shown below (which is annotated based on the regions of the nucleic acid as shown in brackets. In some embodiments, the nucleic acid vector comprises the sequence as shown below without the introns.

### AAV2-ITR

(SEQ ID NO: 3)
TTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGA
GCGAGCGAGCGCGCAGAGAGGGAGTGGCCAACTCCATCACTAGGGGT
TCCT

### B19p6 Promoter

(SEQ ID NO: 7)
CCAACCCTAATTCCGGAAGTCCCGCCCACCGGAAGTGACGTCACAGG
AAATGACGTCACAGGAAATGACGTAATTGTCCGCCATCTTGTACCGG
AAGTCCCGCCTACCGGCGGCGACCGGCGGCATCTGATTTGGTGTCTT
CTTTTAAATTTTAGCGGGCTTTTTTCCCGCCTTATGCAAATGGGCAG
CCATTTTAAGTGTTTTACTATAATTTTATTGGTTAGTTTTGTAACGG
TTAAAATGGGCGGAGCGTAGGCGGGGACTACAGTATATATAGCACGG
CACTGCCGCAGCTCTTTCTTTCTGGGCTGCTTTTTCCTGGACTTTCT
TGCTGTTTTTTGTGAGCTAACTAACAGGTATTTATACTACTTGTTAA
CATACTAA

### Human Anti-Sickling β-Globin Gene

Exon 1
(SEQ ID NO: 10)
ATGGTGCACCTGACTCCTGAGGAGAAGTCTGCCGTTACTGCCCTGTG
GGGCAAGGTGAACGTGGATGAAGTTGGTGGTGAGGCCCTGGGCA
Intron 1
(SEQ ID NO: 11)
GGTTGGTATCAAGGTTACAAGACAGGTTTAAGGAGACCAATAGAAAC
TGGGCATGTGGAGACAGAGAAGACTCTTGGGTTTCTGATAGGCACTG
ACTCTCTCTGCCTATTGGTCTATTTTCCCACCCTTA
Exon 2
(SEQ ID NO: 12)
GGCTGCTGGTGGTCTACCCTTGGACCCAGAGGTTCTTTGAGTCCTTT
GGGGATCTGTCCACTCCTGATGCTGTTATGGGCAACCCTAAGGTGAA
GGCTCATGGCAAGAAAGTGCTCGGTGCCTTTAGTGATGGCCTGGCTC
ACCTGGACAACCTCAAGGGCACCTTTGCC*CAG*CTGAGTGAGCTGC
ACTGTGACAAGCTGCACGTGGATCCTGAGAACTTCAGG
*CAG* = T87Q
Delta 12 Intron (372 bp-deletion in Intron 2)
(SEQ ID NO: 13)
GTGAGTCTATGGGACCCTTGATGTTTTCTTTCCCCTTCTTTTCTATG
GTTAAGTTCATGTCATAGGAAGGGGAGAAGTAACAGGGTACACATAT
TGACCAAATCAGGGTAATTTTGCATTTGTAATTTTAAAAAATGCTTT
CTTCTTTTAATATACTTTTTTGTTTATCTTATTTCTAATACTTTCCC
TAATCTCTTTCTTTCAGGGCAATAATGATACAATGTATCATGCCTCT
TTGCACCATTCTAAAGAATAACAGTGATAATTTCTGGGTTAAGGCAA
TAGCAATATTTCTGCATATAAATATTTCTGCATATAAATTGTAACTG
ATGTAAGAGGTTTCATATTGCTAATAGCAGCTACAATCCAGCTACCA
TTCTGCTTTTATTTTATGGTTGGGATAAGGCTGGATTATTCTGAGTC
CAAGCTAGGCCCTTTTGCTAATCATGTTCATACCTCTTATCTTCCTC
CCACAG
Exon 3
(SEQ ID NO: 14)
CTCCTGGGCAACGTGCTGGTCTGTGTGCTGGCCCATCACTTTGGCAA
AGAATTCACCCCACCAGTGCAGGCTGCCTATCAGAAAGTGGTGGCTG
GTGTGGCTAATGCCCTGGCCCACAAGTATCACTAA
Poly A sequence
(SEQ ID NO: 26)
GCTCGCTTTCTTGCTGTCCAATTTCTATTAAAGGTTCCTTTGTTCCC
TAAGTCCAACTACTAAACTGGGGGATATTATGAAGGGCCTTGAGCAT
CTGGATTCTGCCTAATAAAAAACATTTATTTTCATTGCAATGATGTA
TTTAAATTATTTCTGAATATTTTACTAAAAAGGGAATGTGGGAGGTC
AGTGCATTTAAAACATAAAGAAATGAAGAGCTAGTTCAAACCTTGGG
AAAATACACTATATCTTAAACTCCATGAAAGAAGGTGAGGCTGCAAA
CAGCTAATGCACATTGGCAACAGCCCCTGATGCCTATGCCTTATTCA
TCCCTCAGAAAAGGATTCAAGTAGAGGCTTGATTTGGAGGTTAAAGT
TTTGCTATGCTGTATTTTACATTACTTATTGTTTTAGCTGTCCTCAT
GAATGTCTTTTCACTACCCATTTGCTTATCCTGCATCTCTCAGCCTT
GACTCCACTCAGTTCTCTTGCTTAGAGATACCACCTTTCCCCTGAAG
TGTTCCTTCCATGTTTTACGGCGAGATGGTTTCTCCTCGCCTGGCCA
CTCAGCCTTAGTTGTCTCTGTTGTCTTATAGAGGTCTACTTGAAGAA
GGAAAAACAGGGGGCATGGTTTGACTGTCCTGTGAGCCCTTCTTCCC
TGCCTCCCCCACTCACAGTGACCCGGAATCTGCAGTGCTAGTCTCCC
GGAACTATCACTCTTTCACAGTCTGCTTTGGAAGGACTGGGCTTAGT
ATGAAAAGTTAGGACTGAGAAGAATTTGAAAGGGGGCTTTTTGTAGC
TTGATATTCACTACTGTCTTATTACCCTATCATAGGCCCACCCCAAA
TGGAAGTCCCATTCTTCCTCAGGATGTTTAAGATTAGCATTCAGGAA
GAGATCAGAGGTCTGCTGGCTCCCTTATCATGTCCCTTATGGTGCTT
CTGGCTCTGCAGTTATTAGCATAGTGTTACCATCAACCACCTTAACT
TCATTTTTCTTATTCAATACCTAGCGCGTATCGCGGGATCCACTAGT
TCT
AAV2-ITR
(SEQ ID NO: 27)
AGGAACCCCTAGTGATGGAGTTGGCCACTCCCTCTCTGCGCGCTCGC
TCGCTCACTGAGGCCGCCCGGGCAAAGCCCGGGCGTCGGGCGACCTT
TGGTCGCCCGGCCTCAGTGAGCGAGCGAGCGCGCAGAGAGGGAGTGG
CCAA

In some embodiments of the above sequence, the AAV2 ITRs are replaced with AAV6 ITRs, the B19p6 promoter is replaced with an HS2 enhancer and β-globin promoter, and/or the human anti-sickling β-globin gene is replaced with a human γ-globin gene.

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid containing the nucleic acid vector may be combined with one or more helper plasmids, e.g., that contain a rep gene (e.g., encoding Rep78, Rep68, Rep52 and Rep40) and a cap gene (e.g., encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene (e.g., encoding a rAAV capsid protein as described herein) and a second helper plasmid comprising a E1a gene, a E1b gene, a E4 gene, a E2a gene, and a VA gene. In some embodiments, the rep gene is a rep gene derived from AAV2 or AAV6 and the cap gene is derived from AAV2 or AAV6 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

The disclosure also contemplates host cells that comprise at least one of the disclosed rAAV particles or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cell of erythroid lineage, such as a CD36⁺ burst-forming units-erythroid (BFU-E) cell or a colony-forming unit-erythroid (CFUE-E) progenitor cell.

### Subjects

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of complete blood count, hemoglobin electrophoresis, Fe Binding Capacity, urine urobilin and urobilogen, peripheral blood smear, hematocrit, and genetic testing.

In some embodiments, a host cell is derived from a subject and use to produce a host cell suspension as described herein.

In some embodiments, the subject has or is suspected of having a disease involving blood cells (e.g., a disease caused by a defect, such as a genetic mutation, in one or more blood cell types). Exemplary blood cells include T cell, B cells, dendritic cells, macrophages, monocytes, and hematopoietic stem cells. In some embodiments, the disease is a blood cell cancer, e.g., a leukemia (such as Acute lymphocytic leukemia, Acute myelogenous leukemia, Chronic lymphocytic leukemia, or Chronic myelogenous leukemia), lymphoma (such as Hodgkin lymphoma or non-Hodgkin lymphoma), or myeloma (such as multiple myeloma). Other exemplary diseases involving blood cells include anemia, hemophilia, myelodysplastic syndrome, sickle cell disease, thalassemia, deep vein thrombosis, von Willebrand disease, factor II, V, VII, X, or XII deficiency, Polycythemia vera, thrombocytopenia and Idiopathic thrombocytopenic purpura. Subjects having such diseases can be identified by the skilled practitioner according to methods known in the art, e.g., using one or more of a complete blood count, platelet aggregation test, bleeding time test, genetic testing, and biomarker assays.

In some embodiments, the subject has or is suspected of having cancer. Exemplary cancers include breast cancer, prostate cancer, ovarian cancer, cervical cancer, skin cancer, pancreatic cancer, colorectal cancer, renal cancer, liver cancer, brain cancer, lymphoma, leukemia, myeloma, lung cancer and the like. Subjects having cancer can be identified by the skilled practitioner according to methods known in the art, e.g., using one or more of a biopsy, x-ray, CT scan, Magnetic Resonance Imaging (MRI), ultrasound, genetic testing, and biomarker assays.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1: Recombinant AAV-Parvovirus B19 Hybrid Vectors for Gene Therapy of Human Hemoglobinopathies

The generation of a hybrid human parvovirus containing the adeno-associated virus 2 (AAV2) capsid and the human parvovirus B19 genome was previously described (Proc. Natl. Acad. Sci., USA, 86: 8078-8082, 1989). Subsequently, the parvovirus B19 promoter at map unit 6 (B19p6) was shown to be necessary and sufficient to confer human erythroid cell-tropism to the AAV2-B19p6 hybrid virus (Proc. Natl. Acad. Sci., USA, 92: 12416-12420, 1995). These studies led to the development of AAV2-B19p6 hybrid vectors with which erythroid lineage-restricted transgene expression from the B19p6 promoter could be achieved following stable transduction of murine hematopoietic stem cells (HSCs). In more recent studies, it was observed that of the 10 most commonly used AAV serotypes, AAV6 was the most efficient in transducing human HSCs (Cytotherapy, 15: 986-996, 2013). When the B19p6 promoter-driven expression cassette was encapsidated in tyrosine-mutant AAV6 capsids, erythroid lineage-restricted, high-level expression of the reporter transgene was achieved following stable transduction of human HSCs, both in vitro as well as in a murine xenograft model in vivo (PLoS One, 8: e58757, 2013). More interestingly, the level of the reporter transgene expression from the B19p6 promoter was significantly higher than that from the human β-globin gene promoter.

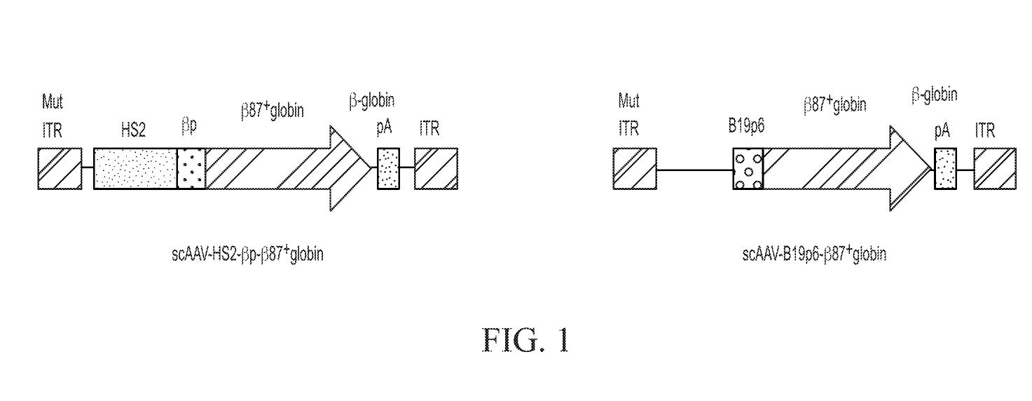

As described herein, recombinant AAV6 was generated containing the human β-globin gene driven by either the B19p6 promoter or the β-globin gene promoter (FIG. 1). Studies are currently underway to determine whether high levels the β-globin protein can be expressed, which would be expected to lead to phenotypic correction of both β-thalassemia and sickle cell disease. The sequence of the B19p6 promoter is provided below:

| (SEQ ID NO: 7) | |
|---|---|
| 1 | CCAACCCTAA TTCCGGAAGT CCCGCCCACC GGAAGTGACG TCACAGGAAA TGACGTCACA |
| 61 | GGAAATGACG TAATTGTCCG CCATCTTGTA CCGGAAGTCC CGCCTACCGG CGGCGACCGG |
| 121 | CGGCATCTGA TTTGGTGTCT TCTTTTAAAT TTTAGCGGGC TTTTTTCCCG CCTTATGCAA |
| 181 | ATGGGCAGCC ATTTTAAGTG TTTTACTATA ATTTTATTGG TTAGTTTTGT AACGGTTAAA |
| 241 | ATGGGCGGAG CGTAGGCGGG GACTACAGTA TATATAGCAC GGCACTGCCG CAGCTCTTTC |
| 301 | TTTCTGGGCT GCTTTTTCCT GGACTTTCTT GCTGTTTTTT GTGAGCTAAC TAACAGGTAT |
| 361 | TTATACTACT TGTTAACATA CTAA |

In this context, it is important to note that the use of recombinant lentiviral vectors in a clinical trial led to transfusion-independence in a young patient with β-thalassemia, but also activated a cellular proto-oncogene, frequently associated with preleukemia (Nature, 467: 318-322, 2010).

Thus, the recombinant AAV6-B19p6-β-globin vectors promise to prove to be a safer alternative for the potential gene therapy of human hemoglobinopathies in general, and β-thalassemia and sickle cell disease in particular.

### Example 2: Development of Chimeric AAV6-B19 Vectors for In Vivo Targeting of Human Hematopoietic Stem Cells

Pathogenic human parvovirus B19, which has a remarkable tropism for primary human erythroid progenitor cells in the bone marrow, utilizes activated a5b1 integrin as a cellular co-receptor to gain entry into target cells (Blood, 102: 3927-3933, 2003), following binding to the erythrocyte P antigen as receptor, which is expressed abundantly on these cells as well as on mature red blood cells (RBCs). However, RBCs lack the expression of a5b1 integrin, and as a consequence, B19 fails to enter these cells, but effectively utilizes mature RBCs to traffic to the bone marrow, where the target erythroid progenitor cells reside.

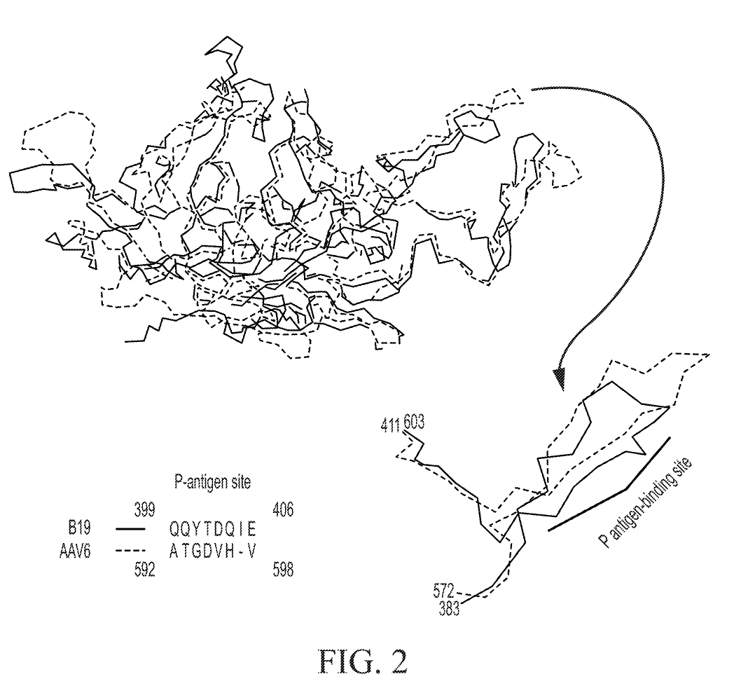

Although of the 10 most commonly used AAV serotypes, AAV6 is the most efficient serotype for transducing primary human hematopoietic stem cells (HSCs), both in vitro and in murine xenograft models in vivo (Cytotherapy, 15: 986-998, 2013; PLoS One, 8(3): e58757, 2013), AAV6 vector promiscuity makes it difficult to target HSCs in vivo. Here, the plan is to exploit the RBC-binding property of B19, mediated by the P antigen-binding site on the B19 capsid, to develop a chimeric AAV-B19 vector with the proven safety and efficacy of AAV6, and the target-specificity of B19, by inserting the P antigen-binding site into the AAV6 capsid. Based on AAV crystal structure, combined with various site-directed and insertion mutagenesis studies of the capsid gene, specific regions of the capsid viral proteins were identified that are surface-exposed and tolerant to insertion of the peptides (FIG. 2). Amino acids in the AAV6 loop VIII (residues 592 to 598) are substituted with that of the B19 P antigen-binding site (residues 399 to 406). Alternatively, the entire loop VIII of AAV6 (residues 572 to 603) is substituted with the entire P antigen-binding site (residues 383 to 411). It is hypothesized that these study will result in development of safe and efficient vectors for targeting primary human HSCs directly in the patient's bone marrow. The current treatment of human hemoglobinopathies involve bone marrow harvest, HSC isolation and purification, ex vivo transduction, and HSC transplantation, using lentivirus-based vectors. The major disadvantages of these treatments include cumbersome procedures, high patient care costs, and the potential risk of initiating preleukemia associated with lentiviral vectors (Nature, 467: 318-322, 2010).

Thus, the ability to deliver the chimeric AAV6-B19 therapeutic vector (e.g., rAAV) directly to the patient's bone marrow to achieve high-efficiency transduction of HSCs should circumvent each of the problems associated with the use of lentiviral vectors. The availability of these novel AAV6-B19 chimeras should prove useful in the potential gene therapy of human hematopoietic disorders in general, and human hemoglobinopathies in particular.

### Example 3: Strategies to Achieve High-Efficiency Transduction of Human Hematopoietic Stem Cells with Recombinant AAV

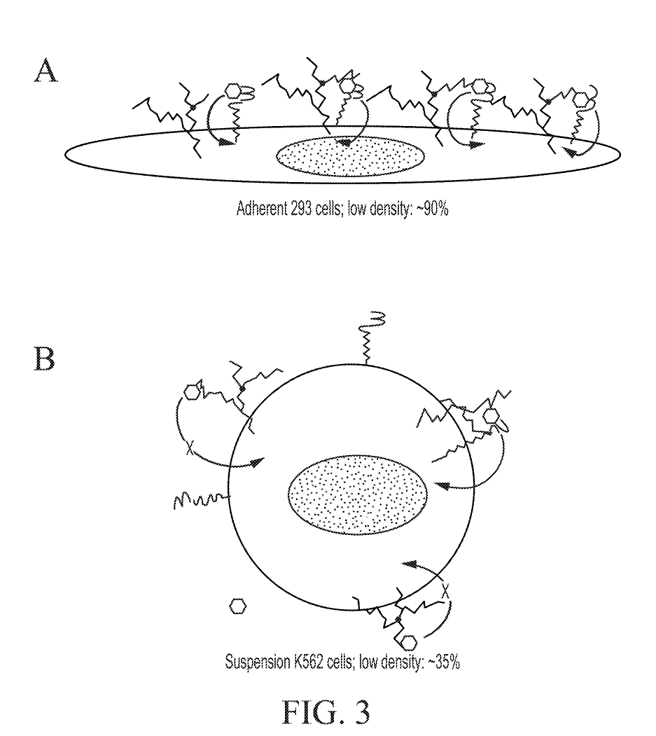

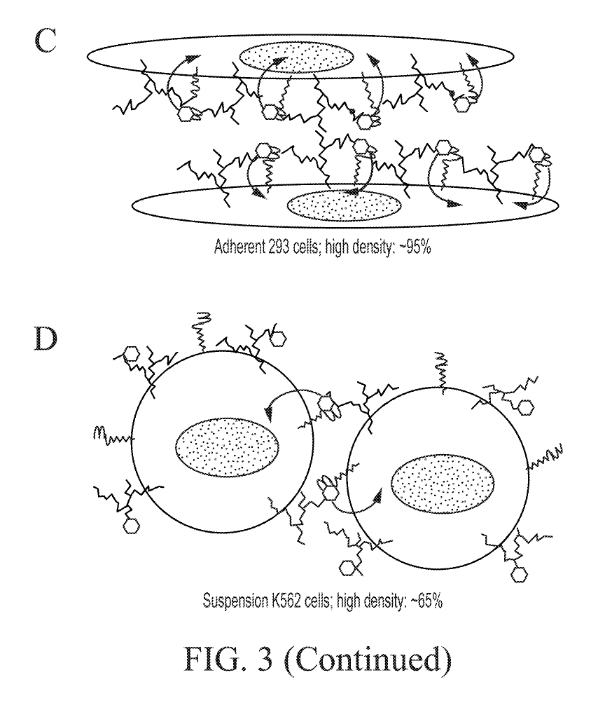

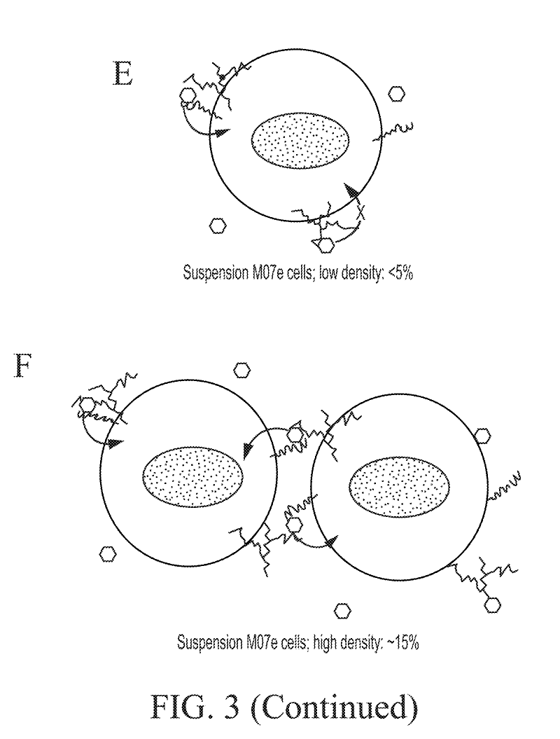

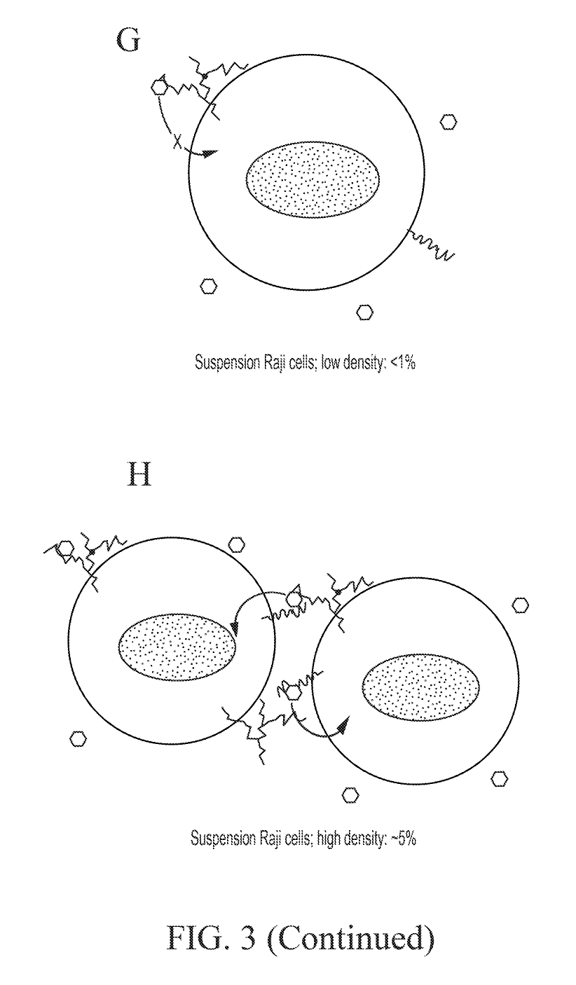

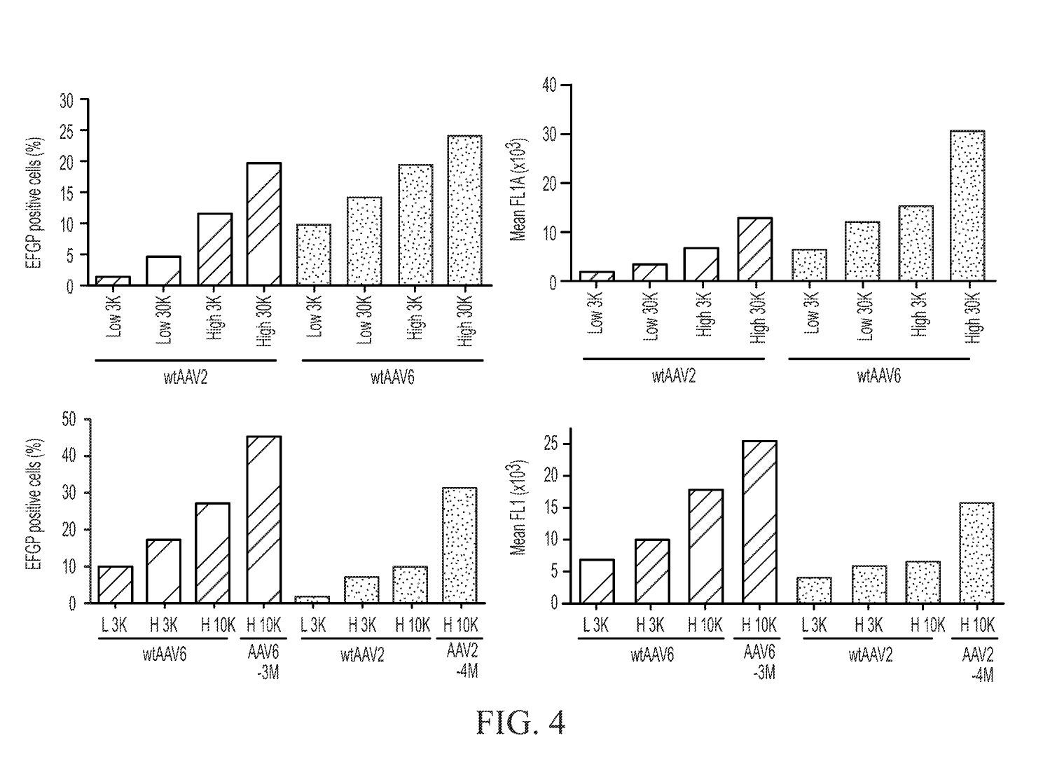

Unlike most adherent cells, human hematopoietic stem cells (HSCs), grown in suspension, are not transduced efficiently by AAV2 serotype vectors (e.g., rAAV), although these cells express both heparin sulfate proteoglycan (HSPG) and Fibroblast growth factor receptor 1 (FGFR1), albeit at low levels. It was reasoned that the lack of proximity of HSPG and FGFR1 on these cells might account for the suboptimal transduction of these cells, and it was hypothesized that if the transduction was performed at high cell density, presumably allowing for HSPG on one cell to come in close proximity to FGFR1 on the neighboring cell, then AAV2 bound to HSPG on one cell could utilize FGFR1 on the neighboring cell to gain entry in the latter, and vice versa, thus leading to increased transduction. To test this hypothesis, primary human HSCs were transduced at either low or high cell density. Whereas only ˜5% of these cells were transduced at low-density, the transduction efficiency increased up to ˜20% at high-density. Thus, these studies have revealed a novel mechanism, which has been termed “cross-transduction” (FIG. 3), in which AAV vectors (e.g., particles) exploit to gain entry into target cells. Of the 10 commonly used AAV serotypes, AAV6 is the most efficient in transducing primary human HSCs, both in vitro and in murine xenograft models in vivo (Cytotherapy, 15: 986-998, 2013; PLoS One, 8(3): e58757, 2013). However, the transduction efficiency of these vectors ranged between ˜6-87% in HSCs obtained from several different donors (n=11). Such a wide range of transduction efficiency of AAV6 vectors is presumably due to different levels of expression of the putative receptors and/or co-receptors on these cells. In the present study, the transduction efficiency of AAV2 vectors could be augmented both by performing transduction of hematopoietic stem cells (HSCs) with the wild-type (wt)-AAV2 vectors at high cell density, or by using capsid-modified Y444F+Y500F+Y731F+T491V-mutant AAV2 vectors. It was examined whether similar strategies could also be employed to increase the transduction efficiency of HSCs from donors that are not transduced efficiently by AAV6 vectors. Primary human HSCs were transduced with AAV6 vectors either at low or at high density. Whereas only ˜14% of the cells transduced at low-density with high multiplicity of infection (MOI) expressed the transgene, the transduction efficiency at high-density increased up to ˜20% and 25%, at low, and high MOIs, respectively, also with a significant increase in the mean fluorescence intensity, thus corroborating that the initial cell-cell contact was a critical factor in achieving increased transduction. Next, the transduction efficiencies of the wild-type (wt) and the capsid-modified triple-mutant (Y705F+Y731F+T491V) AAV6 vectors were compared. Again, the wt- and the capsid-modified quadruple-mutant (Y444F+Y500F+Y731F+T491V) AAV2 vectors were used for comparison. Again, ˜27% transduction efficiency of the wt-AAV6 vectors was increased by up to ˜45% with the capsid-modified AAV6 vectors, with a concomitant increase in the mean fluorescence intensity (FIG. 4).

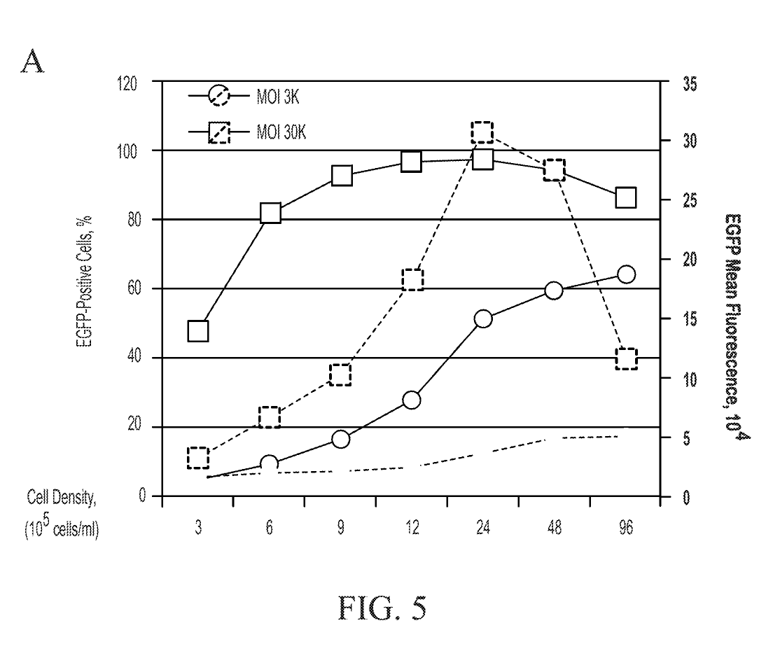

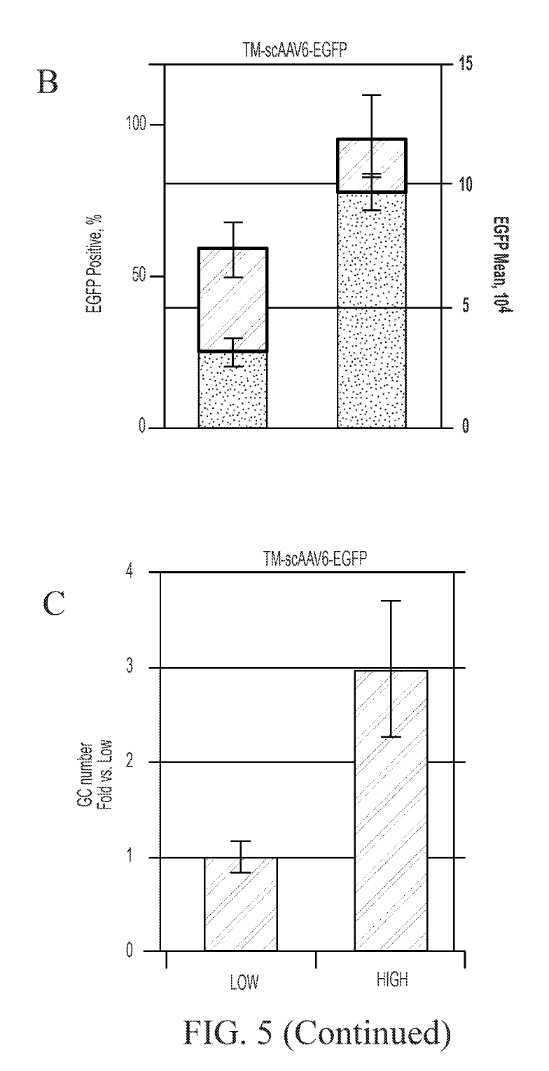

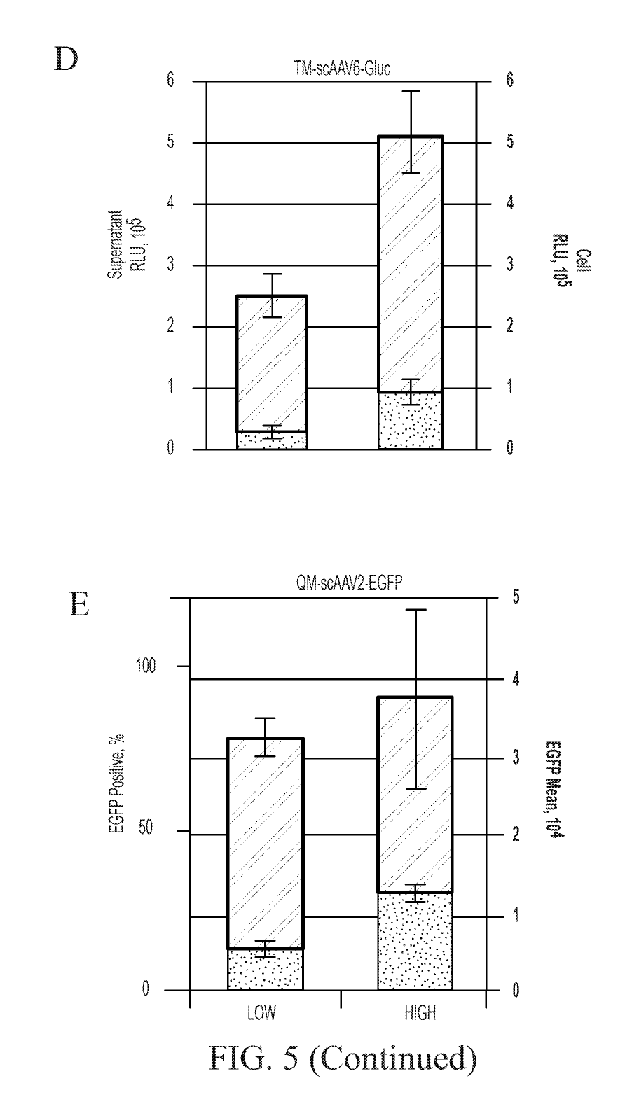

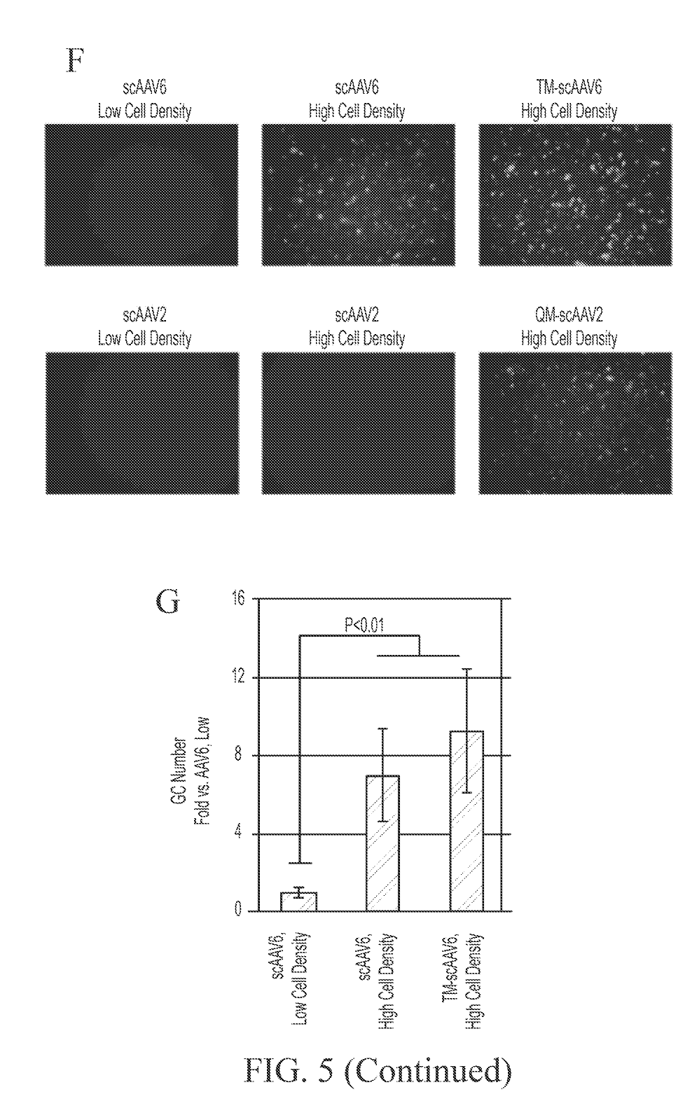

Additional data were obtained from a series of experiments that were performed to further investigate the relationship between cell density and transduction efficiency. The results, as analyzed by flow cytometry 48 hours post-transduction, indicated that, compared to the conventionally used 6×10⁵ cells/mL, increased cell density, up to 1.0×10⁷ cells/mL, dramatically enhanced the scAAV6-mediated transgene expression, in both the EGFP-positivity and EGFP mean fluorescence intensity (FIG. 5A), presumably due to the increased probability of more efficient rAAV attachment to the cell receptor and/or co-receptor. Next, K562 cells were transduced with the optimized TM-scAAV6-CBAp-EGFP vectors either at low-density (1×10⁶ cells/mL) or high-density (1×10⁷ cells/mL). Whereas only ˜25% of K562 cells were transduced at low-density, the transduction efficiency at high-density increased up to 77%, and the EGFP mean value increased to 160% (FIG. 5B). The enhancement of transgene expression also correlated with a significant increase in the intra-cellular viral genome copy number (FIG. 5C), as determined by qPCR of total DNA isolated 2 hours post-transduction. Similar results were obtained with the TM-scAAV6 rAAV expressing the Guassia luciferase (Gluc) transgene (FIG. 5D), as well as when the optimized AAV2-CBAP-EGFP vectors containing the quadruple mutation (Y444F+Y500F+Y731F+T491V; QM-scAAV2) were used (FIG. 5E). Similar results were also obtained when these serotypes were used to transduce HSPCs from a donor, which are transduced extremely poorly under conventional conditions (FIG. 5F). These studies further corroborate the novel mechanism of “cross-transduction” by recombinant AAV of human cells in general, and HSCs in particular, wherein initial cell-cell contact is critical in achieving high-efficiency transduction.

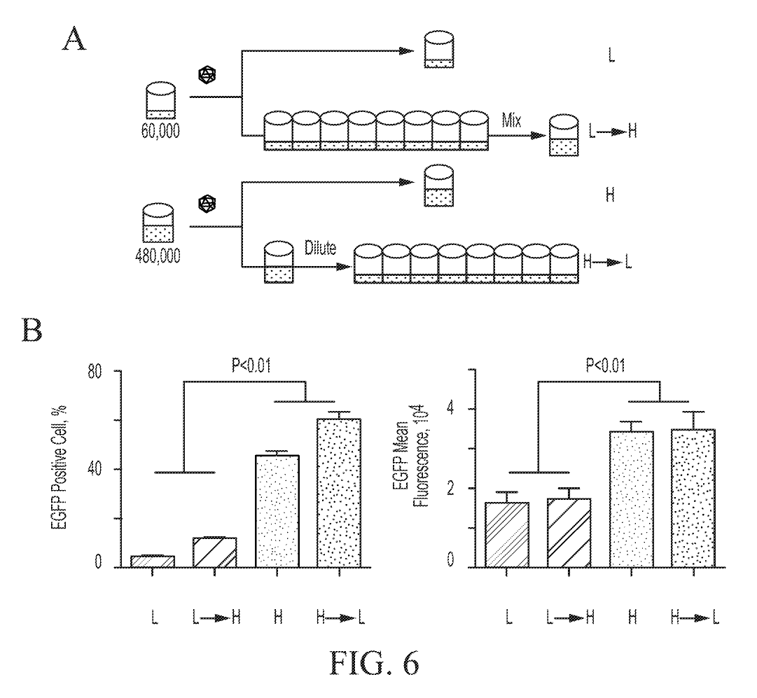

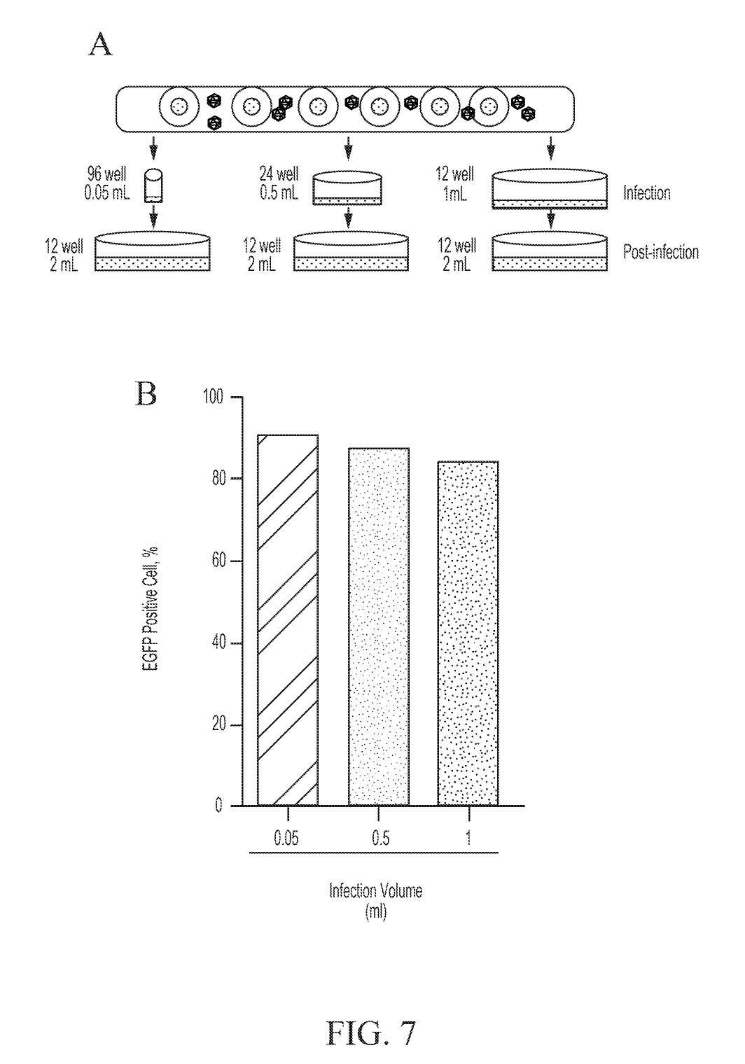

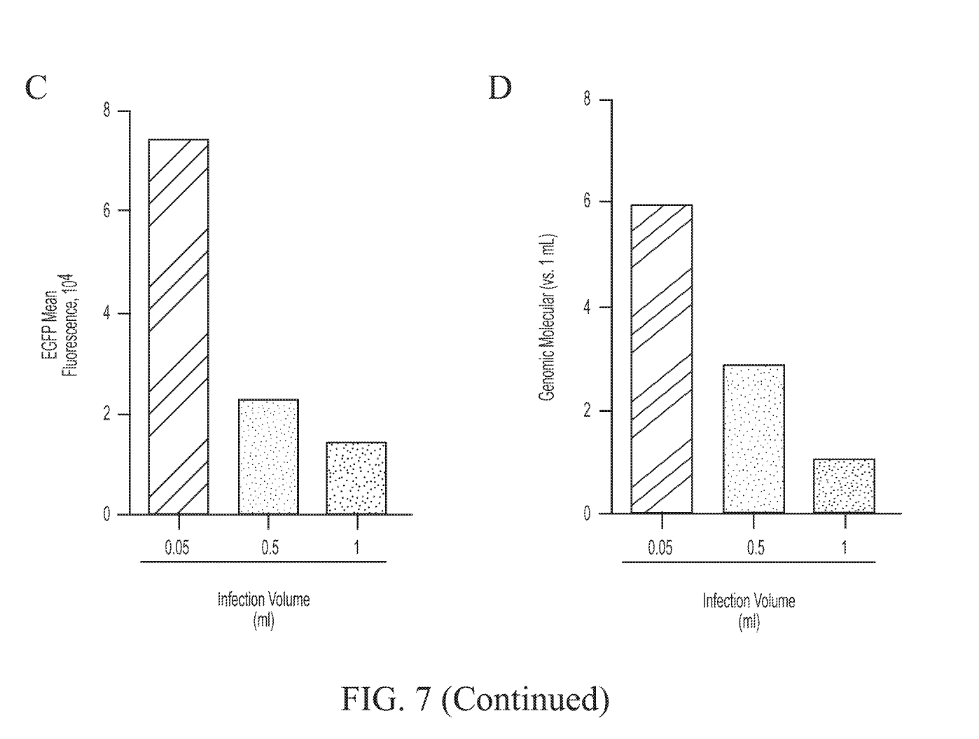

That the initial cell-cell contact was critical in achieving high-efficiency transduction, was further corroborated by experiments in which cells were transduced at low-density, and subsequently pooled together to reach high-density, and conversely, cells were transduced at high-density, and soon after transduction, were diluted to low-density (FIG. 6A). The increased transduction was observed only under the latter condition (FIG. 6B). In the second set of experiments, a fixed number of K562 cells were infected with rAAV in various volume for 2 hours and subsequently diluted in the same volume of 2 mL (FIG. 7A). Once again, the increased transduction efficiency was observed only under the condition of high cell density (FIG. 7B, 7C), accompanied with a significantly increased intra-cellular viral genome copy numbers (FIG. 7D).

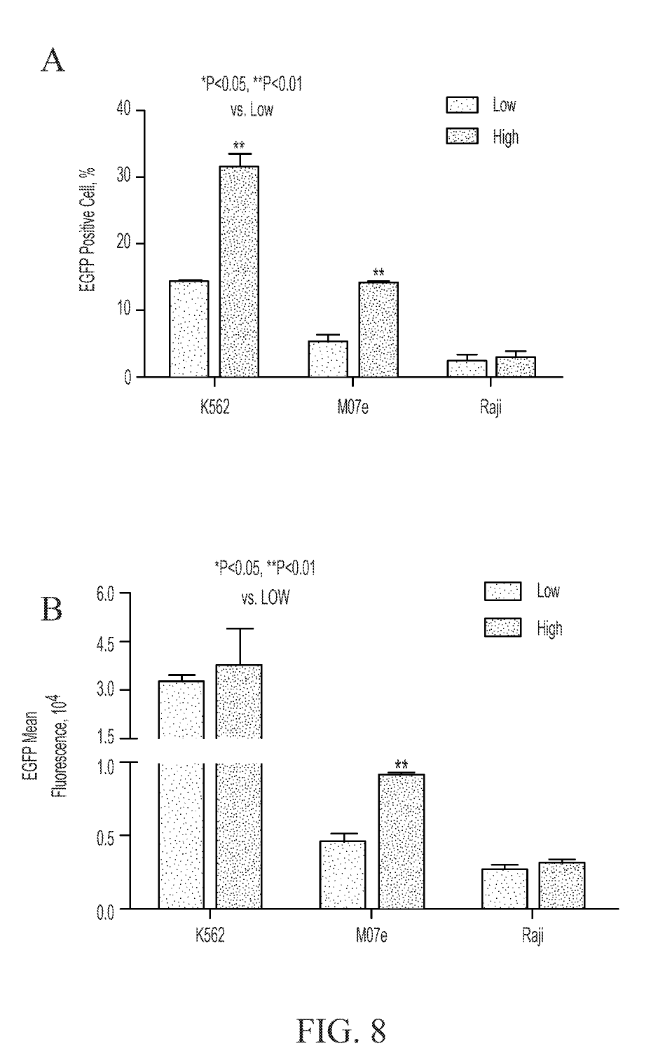

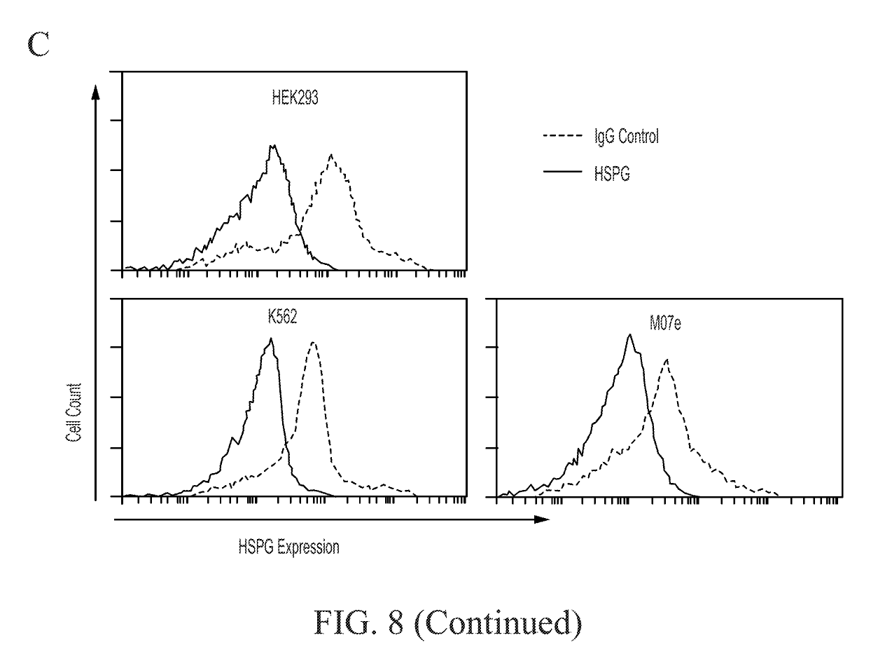

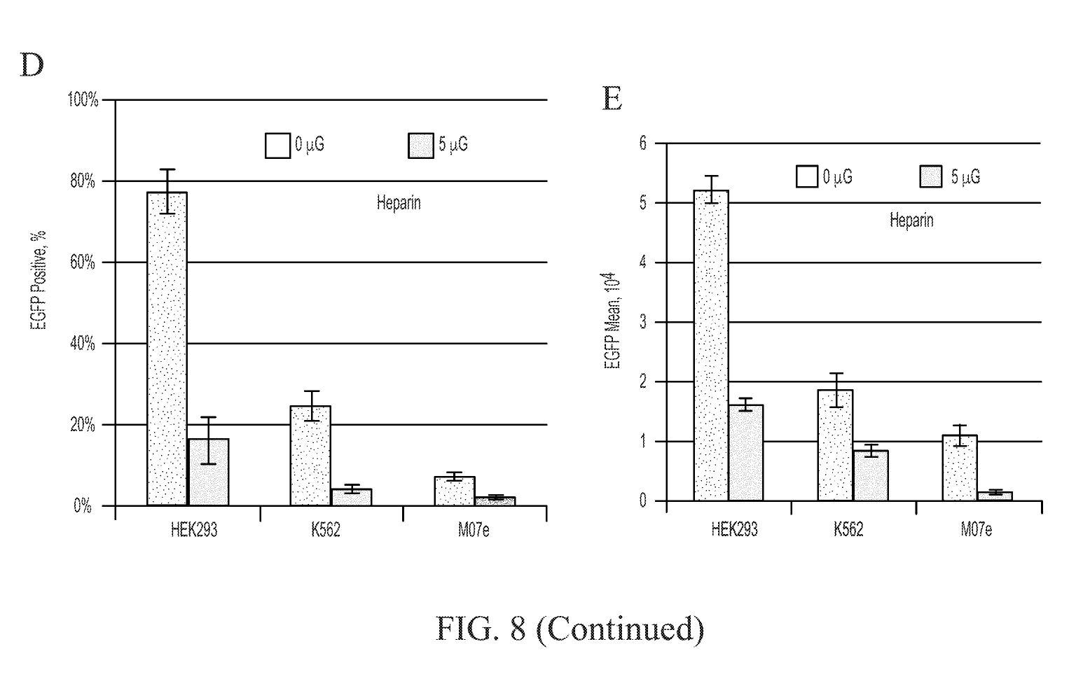

These studies were extended to include two additional human hematopoietic cell lines, M07e and Raji, which express low to extremely low levels of heparin sulfate proteoglycan (HSPG), the primary receptor for AAV2, and consequently, are transduced extremely poorly by AAV2. Under the condition of high cell density, significantly enhanced transduction of M07e cells, but not Raji cells, was observed (FIG. 8A, 8B), since M07e cells express high levels of AAV2 co-receptor and fibroblast growth factor receptor 1 (FIG. 8C). Raji cells, by comparison, express undetectable levels of both HSPG and FGFR117. To address the possibility whether alternative receptors/co-receptors were being used under the condition of high cell density, K562 cells were transduced with scAAV2 in the absence or the presence of heparin, which is known to compete for AAV2 cellular entry. Heparin at 5 μg/mL significantly reduced the transduction efficiency of scAAV2 under the condition of high cell density for each of the cell types tested (FIG. 8D, 8E). These results strongly suggest that the putative receptors/co-receptors for viral entry remain unaltered under the condition of high cell density.

The efficacy of AAV-mediated transduction of primary HSPCs derived from bone marrow (BM) as well as from umbilical cord blood (CB) was further evaluated. BM-derived CD34+ cells from individual donors (or a mixture from 10 donors) were purchased form a commercial source (AllCells, LLC, Alameda, Calif., USA), and were used to transduce with different scAAV-CBAp-EGFP at an MOI of 10,000 vgs/cell without fetal bovine serum (FBS). Transgene expression was evaluated by flow cytometry 48 hours post-transduction. As shown in Table 2, consistent with our previously published studies, whereas scAAV6 transduced human HSPCs more efficiently than scAAV2, capsid modification on both rAAVs further enhanced their transduction efficiency. The transgene expression at high cell density was consistently higher than that at low cell density. The increased transduction efficiency in human HSPCs at high cell density also correlated with a significantly increased intra-cellular viral genome copy number 2 hours post-viral transduction. However, the extent of transgene expression declined over time, and in none of the cell populations tested, the viral genome copy number was below the detection limit of qPCR 14 days post-transduction.

TABLE 2
Transduction efficiency of AAV in primary human HSPCs
from various donors.

| | WT- | WT- | TM- | WT- | QM- | WT- | TM- |
|---|---|---|---|---|---|---|---|
| | AAV2 | AAV6 | AAV6- | AAV2 | AAV2- | AAV6 | AAV6- |
| | Low Cell Density | | | High Cell Density | | | |
| Do- | ND | 1.3% | ND | ND | ND | 19.4% | ND |
| nor | | 5,090 | | | | 9,080 | |
| #1 | | | | | | | |
| Do- | 1.4%, | 9.8% | ND | 7.2% | 31.1% | 27.0% | 45.2% |
| nor | 4184 | 6904 | | 5810 | 15672 | 9994 | 25373 |
| #2 | | | | | | | |
| Do- | ND | ND | 28.6%, | ND | ND | ND | 58.5% |
| nor | | | 8,246 | | | | 23,846 |
| #3 | | | | | | | |
| Do- | 4.4%, | 9.5%, | ND | 11.6%, | 19.6%, | 23.9%, | ND |
| nor | 3,720 | 6,512 | | 6,686 | 13,001 | 15,434 | |
| #4 | | | | | | | |

Data are presented as % EGFP positive cells, and EGFP mean fluorescence intensities.
ND = Not done.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant AAV (rAAV) particle comprising a nucleic acid vector comprising a parvovirus B19p6 promoter operatively linked to a heterologous gene, wherein the rAAV particle is not AAV2.

2. The rAAV particle of any prior claim, wherein the heterologous gene is a globin gene.

3. The rAAV particle of claim 2, wherein the globin gene is selected from the group consisting of a β-globin gene, an anti-sickling β-globin gene, and a γ-globin gene.

4. The rAAV particle of any prior claim, wherein the globin gene is a human globin gene.

5. The rAAV particle of any prior claim, wherein the globin gene is a human β-globin gene or human anti-sickling β-globin gene.

6. The rAAV particle of any prior claim, wherein the rAAV particle is a AAV6 particle.

7. The rAAV particle of claim 6, wherein the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination thereof.

8. The rAAV particle of claim 7, wherein the modified capsid protein comprises a non-tyrosine residue and/or a non-threonine residue at one or more of or each of Y705, Y731, and T492 of a wild-type AAV6 capsid protein.

9. The rAAV particle of claim 7 or 8, wherein the non-tyrosine residue is phenylalanine and the non-threonine residue is valine.

10. The rAAV particle of any prior claim, wherein the nucleic acid vector further comprises AAV2 or AAV6 inverted terminal repeat sequences (ITRs) flanking the parvovirus B19p6 promoter operatively linked to the heterologous gene.

11. A nucleic acid vector comprising a parvovirus B19p6 promoter operatively linked to a globin gene.

12. The nucleic acid vector of any prior claim, wherein the globin gene is selected from the group consisting of a β-globin gene, an anti-sickling β-globin gene, and a γ-globin gene.

13. The nucleic acid vector of any prior claim, wherein the globin gene is a human globin gene.

14. The nucleic acid vector of any prior claim, wherein the globin gene is a human β-globin gene or human anti-sickling β-globin gene.

15. The nucleic acid vector of any prior claims, wherein the vector further comprises inverted terminal repeats (ITRs) flanking the parvovirus B19p6 promoter operatively linked to the globin gene.

16. A method of targeting gene expression to a cell of erythroid lineage in a subject, the method comprising administering the rAAV particle of any prior claim or the nucleic acid vector of any prior claim to a subject.

17. The method of the prior claim, wherein the subject is a human subject.

18. The method of the prior claim, wherein the cell of erythroid lineage is a hematopoietic stem cell.

19. The method of the prior claim, wherein the cell of erythroid lineage is a CD36⁺ burst-forming units-erythroid (BFU-E) cell or a colony-forming unit-erythroid (CFUE-E) progenitor cell.

20. A method of treating a hemoglobinopathy, the method comprising administering the rAAV particle of any prior claim or the nucleic acid vector of any prior claim to a subject having a hemoglobinopathy.

21. The method of the prior claim, wherein the subject is a human subject.

22. The method of the prior claim, wherein the hemoglobinopathy is β-thalassemia or sickle cell disease.

23. An rAAV capsid protein comprising one or more amino acid substitutions that result in increased P antigen binding compared to a corresponding un-mutated AAV capsid protein, wherein the one or more amino acid substitutions are in a surface exposed loop of the capsid protein.

24. An rAAV capsid protein comprising one or more amino acid substitutions that introduce a P antigen binding site into a surface exposed loop of the capsid protein.

25. The rAAV capsid protein of any one of claims 23-24, wherein the surface exposed loop is loop VIII.

26. The rAAV capsid protein of any one of claims 23-25, wherein a surface exposed loop is replaced by a B19 P antigen binding site.

27. The rAAV capsid protein of claim 26, wherein the B19 P antigen binding site comprises the amino acid sequence QQYTDQIE (SEQ ID NO: 1).

28. The rAAV capsid protein of any one of claims 23-27, wherein the rAAV capsid protein is a variant of an AAV6 capsid protein.

29. A method of increasing rAAV tropism for hematopoietic stem cells, the method comprising altering a surface exposed loop of an AAV capsid protein to introduce one or more amino acid substitutions that result in increased P antigen binding compared to a corresponding un-mutated AAV capsid protein.

30. A method of increasing rAAV tropism for hematopoietic stem cells, the method comprising altering a surface exposed loop of an AAV capsid protein to introduce one or more amino acid substitutions that introduce a P antigen binding site into a surface exposed loop of the capsid protein.

31. The method of any one of claims 29-30, wherein the surface exposed loop is loop VIII.

32. The method of any one of claims 29-31, wherein a surface exposed loop is replaced by a B19 P antigen binding site.

33. The method of claim 32, wherein the B19 P antigen binding site comprises the amino acid sequence QQYTDQIE (SEQ ID NO: 1).

34. The method of any one of claims 29-33, wherein the rAAV capsid protein is a variant of an AAV6 capsid protein.

35. A method of delivering an rAAV to a cell, the method comprising administering an rAAV particle comprising an rAAV capsid protein of any one of claims 23-34 to a subject.

36. The method of claim 35, wherein the cell is a hematopoietic stem cell, a megakaryocyte, an endothelial cell, a cardiomyocyte, a hepatocyte, or a trophoblast.

37. The method of claim 36, wherein the cell is a hematopoietic stem cell.

38. The method of any one of claims 35 to 37, wherein the subject is a human subject.

39. An rAAV particle comprising an rAAV capsid protein of any one of claims 23-38.

40. A nucleic acid encoding an rAAV capsid protein of any one of claims 23-39.

41. The nucleic acid of claim 40, wherein the nucleic acid is a plasmid.

42. A method for efficient AAV transduction of a host cell suspension, the method comprising contacting a host cell suspension with a recombinant AAV (rAAV) particle composition, wherein the host cell suspension has a density of greater than 4,000 cells per microliter.

43. The method of claim 42, wherein the rAAV particle composition is a AAV2 or AAV6 particle composition.

44. The method of any one of claims 42-43, wherein the rAAV particle composition contains 3×10³-1×10⁴ vector genomes(vg)/mL of rAAV particles.

45. The method of any one of claims 42-44, wherein the recombinant AAV (rAAV) particle within the composition comprises a nucleic acid vector that encodes a therapeutic protein.

46. The method of any one of claims 42-45, wherein the recombinant AAV (rAAV) particle within the composition comprises a modified capsid protein comprising a non-tyrosine residue at a position that corresponds to a surface-exposed tyrosine residue in a wild-type AAV2 or AAV6 capsid protein, a non-threonine residue at a position that corresponds to a surface-exposed threonine residue in the wild-type AAV2 or AAV6 capsid protein, a non-lysine residue at a position that corresponds to a surface-exposed lysine residue in the wild-type AAV2 or AAV6 capsid protein, a non-serine residue at a position that corresponds to a surface-exposed serine residue in the wild-type AAV2 or AAV6 capsid protein, or a combination thereof.

47. The method of claim 46, wherein the modified capsid protein comprises a non-tyrosine residue and/or a non-threonine residue at one or more of or each of Y705, Y731, and T492 of a wild-type AAV6 capsid protein.

48. The method of claim 46, wherein the modified capsid protein comprises a non-tyrosine residue and/or a non-threonine residue at one or more of or each of Y444, Y500, Y731, and T491 of a wild-type AAV2 capsid protein.

49. The method of claim 47 or 48, wherein the non-tyrosine residue is phenylalanine and the non-threonine residue is valine.

50. The method of any one of claims 42-49, wherein the host cell suspension comprises stem cells.

51. The method of any one of claims 42-50, wherein the host cell suspension comprises human cells.

52. The method of any one of claims 42-51, wherein the host cell suspension comprises hematopoietic stem cells.

53. The method of any one of claims 42-52, wherein the host cell suspension is a non-adherent host cell suspension.

54. The method of any one of claims 42-53, wherein the method further comprises administering host cells of the host cell suspension to a subject.

55. The method of any one of claims 42-54, wherein host cells of the host cell suspension are obtained from a subject.


SEQUENCE LISTING (28 sequences)

SEQ ID NO: 1 - protein, 8 aa, Human parvovirus B19
QQYTDQIE

SEQ ID NO: 2 - protein, 331 aa, Homo sapiens
MASALWTVLPSRMSLRSLKWSLLLLSLLSFFVMWYLSLPHYNVIERVNWMYFYEYEPIYRQDFHFTLREHSNCSHQNPFLVILVTSHPSDVKARQAIRVTWGEKKSWWGYEVLTFFLLGQEAEKEDKMLALSLEDEHLLYGDIIRQDFLDTYNNLTLKTIMAFRWVTEFCPNAKYVMKTDTDVFINTGNLVKYLLNLNHSEKFFTGYPLIDNYSYRGFYQKTHISYQEYPFKVFPPYCSGLGYIMSRDLVPRIYEMMGHVKPIKFEDVYVGICLNLLKVNIHIPEDTNLFFLYRIHLDVCQLRRVIAAHGFSSKEIITFWQVMLRNTTCHY

SEQ ID NO: 3 - DNA, 145 bp, Adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 4 - DNA, 146 bp, Adeno-associated virus 3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 5 - DNA, 167 bp, Adeno-associated virus 5
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 6 - DNA, 142 bp, Adeno-associated virus 6
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 7 - DNA, 384 bp, Human parvovirus B19
ccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggttagttttgtaacggttaaaatgggcggagcgtaggcggggactacagtatatatagcacggcactgccgcagctctttctttctgggctgctttttcctggactttcttgctgttttttgtgagctaactaacaggtatttatactacttgttaacatactaa

SEQ ID NO: 8 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 9 - protein, 147 aa, Homo sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 10 - DNA, 91 bp, Homo sapiens
atggtgcacctgactcctgaggagaagtctgccgttactgccctgtggggcaaggtgaacgtggatgaagttggtggtgaggccctgggca

SEQ ID NO: 11 - DNA, 130 bp, Homo sapiens
ggttggtatcaaggttacaagacaggtttaaggagaccaatagaaactgggcatgtggagacagagaagactcttgggtttctgataggcactgactctctctgcctattggtctattttcccaccctta

SEQ ID NO: 12 - DNA, 224 bp, Homo sapiens
  misc_feature (171)..(173): T87Q mutation
ggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgcccagctgagtgagctgcactgtgacaagctgcacgtggatcctgagaacttcagg

SEQ ID NO: 13 - DNA, 476 bp, Homo sapiens
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 14 - DNA, 129 bp, Homo sapiens
ctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 15 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFAQLSELHCDKLHVDPENFRLLGNVLVCVLAHHFGKEFTPPVQAAYQKVVAGVANALAHKYH

SEQ ID NO: 16 - protein, 736 aa, Homo sapiens
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - protein, 735 aa, Homo sapiens [sequence omitted]

SEQ ID NO: 18 - protein, 736 aa, Homo sapiens
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 19 - protein, 734 aa, Homo sapiens
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 20 - protein, 724 aa, Homo sapiens
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 736 aa, Homo sapiens
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - protein, 737 aa, Homo sapiens
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 23 - protein, 738 aa, Homo sapiens
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - protein, 736 aa, Homo sapiens
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 738 aa, Homo sapiens
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 26 - DNA, 1037 bp, Homo sapiens
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctctgcagttattagcatagtgttaccatcaaccaccttaacttcatttttcttattcaatacctagcgcgtatcgcgggatccactagttct

SEQ ID NO: 27 - DNA, 145 bp, Homo sapiens
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 28 - protein, 7 aa, Adeno-associated virus 6
ATGDVHV